(19) United States
(12) Patent Application Publication
(10) Pub. No.: US 2008/0075793 A1
(43) Pub. Date: Mar. 27, 2008

(54) ANTIVIRAL COMPOSITIONS AND METHODS OF USE

(76) Inventors: Wayne K. Dunshee, Maplewood, MN (US); Steven S. Kantner, St. Paul, MN (US)

Correspondence Address: 3M INNOVATIVE PROPERTIES COMPANY, PO BOX 33427, ST. PAUL, MN, 55133-3427, US

(21) Appl. No.: 11/534,154

(22) Filed: Sep. 21, 2006

(51) Int. Cl. A61K 36/61; A61K 36/53; A61K 36/28; A61K 36/13; A61K 31/045

(52) U.S. Cl. 424/725; 424/745; 424/742; 424/769; 424/770; 514/729; 424/764

(57) ABSTRACT

Antiviral compositions, especially those useful when applied topically, particularly to mucosal tissues (i.e., mucous membranes), including, in particular, an antiviral lipid component, such as a fatty acid ester, fatty ether, or alkoxide derivative thereof, and an organoleptic neutralizing agent. Such compositions provide effective topical antimicrobial activity and are accordingly useful in the treatment and/or prevention of conditions that are caused, or aggravated by, microorganisms (including viruses).

### BACKGROUND

The use of antimicrobial agents (e.g., antibiotics, antiseptics) plays an important part in current medical therapy. This is particularly true in the fields of dermatology as well as skin and wound antisepsis, where the most effective course of treatment for skin or mucous membranes, which are afflicted with bacterial, fungal, or viral infections or lesions, frequently includes the use of a topical antimicrobial agent.

Dermal afflictions caused by viral infections, such as cold sores and shingles, originate from inside the body. Infections caused by the herpes virus (e.g., herpes simplex virus 1 or 2, referred to as “HSV”), commonly known as “fever blisters” or “cold sores,” affect a majority of the human population. Approximately 80% of American adults are infected with HSV-1, and an estimated 20-40% of adults suffer from recurrent outbreaks as described in Higgins C R, et al., Natural History, management and complications of herpes labialis, J. Med. Virol. 1 (Suppl.):22-26, 1993. Many topical compositions containing known antiviral compounds for use in and around the mouth may relieve the symptoms of infections caused by the herpes virus such as pain, inflammation and/or itchiness often associated with the dermal viral infection or skin lesion.

The use of antiviral lipid components in topical compositions for the treatment of viral infections is disclosed in U.S. Patent Publication No. 2005/0089539-A1 and U.S. application Ser. No. 11/077,864. Formulations containing moderate to high levels of antiviral lipid components, such as fatty acid monoesters of polyhydroxyalcohols, have been shown to effectively minimize the duration and severity of type I Herpes Simplex viral (cold sore) infections occurring in and around the mouth. The antiviral lipid components however have a soapy or bitter taste that lingers in the mouth. Such unpleasant tastes may lead to lower patient compliance resulting in increased duration and severity to the cold sore episode.

A need exists for effective topical compositions containing antiviral lipid components that have improved organoleptic properties.

### SUMMARY OF THE INVENTION

The present invention provides antiviral compositions and methods of using and making the compositions. Compositions comprising an antiviral lipid component and one or more organoleptic neutralizing agents are disclosed that provide effective reduction, inhibition, prevention, or elimination of microbes, particularly viruses, while at the same time providing acceptable palatability in the mouth of a user. These organoleptic neutralizing agents, when combined with the antiviral lipid components that exhibit undesirable taste, effectively neutralize the undesirable taste properties of the antiviral lipid components, resulting in organoleptically acceptable compositions. The organoleptic neutralizing agents may themselves have an undesirable taste. These organoleptic neutralizing agents, many of them having antiseptic properties themselves, also appear to enhance the effectiveness of the antiviral lipid component, providing improved antiviral benefit and broader antimicrobial performance relative to either component applied alone.

Compositions disclosed herein provide effective topical antiviral activity and are accordingly useful in the local treatment and/or prevention of conditions that are caused, or aggravated by, viruses when applied topically to skin or mucosal tissues (i.e., mucous membranes) in and surrounding the oral cavity. The compositions also provide reduction or prevention of lesions caused by viruses, resulting in clinical improvement.

Certain embodiments of the present invention also provide effective reduction, prevention, or elimination of other microbes including bacteria and fungi and hence can be can be particularly useful at treating secondary bacterial or fungal infections that often accompany the primary viral infection.

Significantly, certain embodiments of the present invention have a very low potential for generating microbial resistance. Thus, such compositions can be applied multiple times over one or more days to treat topical infections or to eradicate unwanted bacteria. Furthermore, compositions of the present invention can be used for multiple treatment regimens on the same patient without the fear of generating antimicrobial resistance.

Also, preferred compositions of the present invention have a generally low irritation level for skin, skin lesions, and mucosal membranes.

Compositions of the claimed invention include an antiviral lipid component. In certain embodiments, the antiviral lipid component includes a fatty acid ester of a polyhydric alcohol, a fatty ether of a polyhydric alcohol, a fatty alcohol ester of a hydroxyacid, alkoxylated derivatives thereof (of either the fatty acid ester, ether, or fatty alcohol ester), or combinations thereof. Certain of these antiviral lipids appear to have the ability to migrate through the stratum corneum, providing antiviral activity deeper into the skin that just at the surface.

Compositions of the claimed invention also include one or more organoleptic neutralizing agents. The organoleptic neutralizing agents comprise compounds with a structure selected from the group consisting of a hydrocarbon monoterpene of formula C₁₀H₁₆ selected from an acyclic compound, a monocyclic compound, or a bicyclic compound;

an oxygen containing monoterpene of formula C₁₀H₁₈O selected from an acyclic compound, a monocyclic compound, or a bicyclic compound; an oxygen containing acyclic monoterpene of formula C₁₀H₂₀O; the sesquiterpene patchoulol; the diterpene forskolin; the acetate esters of those oxygenated compounds that are alcohols; and mixtures thereof. The organoleptic neutralizing agents are present in an amount sufficient to neutralize the taste of a antimicrobial lipid formulation.

Compounds containing the structures listed above include myrcene, limonene, beta-phellandrene, alpha-terpinene, gamma-terpinene, alpha pinene, beta-pinene, geraniol, linalool, citronellal, terpinen-4-ol, borneol, 1,8-cineol, isoborneol, and citronellol.

The organoleptic neutralizing agents are typically added to the composition in the form of essential oils. The essential oils that function as organoleptic neutralizing agents contain a major amount of one or more of the compounds listed above. Essential oils containing these compounds include but are not limited to tea tree oil, rosemary oil, lavender, pine oil, myrtle, eucalyptus, citronella, patchouli, and coleus extract oil.

Certain compositions further include an external analgesic component to provide relief to symptoms, such as pain and/or itch relief. Other components that can be included as well are thickeners, moisturizers including emollients and humectants, skin protectants, flavorants, other cosmetic or pharmaceutical actives, and surfactants.

Importantly, compositions of the present invention are capable of destroying microorganisms on or in mammalian tissue. Therefore, concentrations of components employed are generally greater than those that have been used to simply preserve certain topically applied compositions, i.e., prevent the growth of microorganism in topical compositions for purposes other than antisepsis.

In one embodiment, a method of treating a viral infection caused by the herpes virus in or on the skin or mucous membrane is provided, the method comprising contacting the affected area with an antiviral composition comprising an effective amount of an antiviral lipid component comprising a (C7-C12) saturated fatty acid ester of a polyhydric alcohol, a (C8-C22) unsaturated fatty acid ester of a polyhydric alcohol, an alkoxylated derivative thereof, or combinations thereof, wherein the alkoxylated derivative has less than 5 moles of alkoxide per mole of polyhydric alcohol; and an organoleptic neutralizing agent.

In another embodiment, a topical antiviral composition is provided that comprises an antiviral lipid component comprising a (C7-C14) saturated fatty acid monoester of a polyhydric alcohol, a (C8-C22) unsaturated fatty acid monoester of a polyhydric alcohol, an alkoxylated derivative thereof, or combinations thereof, present in an amount greater than 5 wt-% based on the total weight of the composition; and an organoleptic neutralizing agent.

In another embodiment, a method of treating herpes lesions on or in the skin or mucous membranes is provided, the method comprising contacting the affected area with an antiviral composition comprising an effective amount of an antiviral lipid component comprising a (C7-C12) saturated fatty acid ester of a polyhydric alcohol, a (C8-C22) unsaturated fatty acid ester of a polyhydric alcohol, an alkoxylated derivative thereof, or combinations thereof, wherein the alkoxylated derivative has less than 5 moles of alkoxide per mole of polyhydric alcohol; and an organoleptic neutralizing agent.

In a further embodiment, a method of treating and/or preventing a viral infection on mammalian tissue is provided, the method comprising contacting the mammalian tissue with an antiviral composition in an amount effective to kill or inactivate one or more microorganisms, wherein the antiviral composition comprises an effective amount of an antiviral lipid component comprising a (C7-C12) saturated fatty acid ester of a polyhydric alcohol, a (C8-C22) unsaturated fatty acid ester of a polyhydric alcohol, a (C7-C12) saturated fatty ether of a polyhydric alcohol, a (C8-C22) unsaturated fatty ether of a polyhydric alcohol, a (C7-C14) saturated fatty alcohol monoester of a (C2-C8) hydroxycarboxylic acid, a (C8-C22) mono- or poly-unsaturated fatty alcohol monoester of a (C2-C8) hydroxycarboxylic acid, an alkoxylated derivative thereof, or combinations thereof, wherein the alkoxylated derivative has less than 5 moles of alkoxide per mole of polyhydric alcohol; and an organoleptic neutralizing agent.

In most embodiments, the antiviral lipid component is present in an amount of greater than 5 wt-%, more preferably greater than 10 wt-%, even more preferably greater than 15 wt-%, and even more preferably greater than 20 wt-%. Unless otherwise specified, all weight percents are based on the total weight of a “ready to use” or “as used” composition. Preferably, if the antiviral lipid component includes a monoester of a polyhydric alcohol, a monoether of a polyhydric alcohol, or an alkoxylated derivative thereof, then there is no more than 50 wt-%, more preferably no more than 40 wt-%, even more preferably no more than 25 wt-%, and even more preferably no more than 15 wt-% of a diester, diether, triester, triether, or alkoxylated derivative thereof present, based on the total weight of the antiviral lipid component.

In a preferred embodiment, the antiviral lipid component includes a (C8-C12) fatty acid monoester of propylene glycol. In most embodiments the antiviral lipid component comprises propylene glycol monolaurate, propylene glycol monocaprate, propylene glycol monocaprylate, and combinations thereof.

In most embodiments, the antiviral composition includes an external analgesic. Safe and effective external analgesics include those selected from the amine and “caine” type, those selected from the alcohols and ketones type, those selected from the antihistamine type, those selected from hydrocortisone preparations, and mixtures thereof. When used in an appropriate wt-%, they temporary relieve the symptoms, such as pain or itch, associated with the viral infection. Preferred amine and “caine” type external analgesics include benzocaine, butamben picrate, dibucaine (or dibucaine HCl), dimethisoquin HCl, dyclonine HCl, lidocaine (or lidocaine HCl), pramoxine HCl, tetracaine (or tetracaine HCl), and mixtures thereof. Preferred alcohol and ketone type external analgesics include benzyl alcohol, camphor, camphorated metacresol, juniper tar, menthol, phenol, phenolate sodium, resorcinol, and mixtures thereof. Preferred antihistamine type external analgesics include diphenhydramine HCl, tripelennamine HCl, and mixtures thereof. Preferred hydrocortisone preparations include hydrocortisone, hydrocortisone acetate, and mixtures thereof. Mixtures of external analgesics from more than one type are also useful. Further information concerning safe and effective analgesics is provided in the Tentative Final Monograph on External Analgesic Drug Products for Over-the-counter Human Use, published by the United States Food and Drug Administration in the Federal Register, Volume 48, Number 27, Feb. 8, 1983, pages 5852 to 5869.

In certain embodiments, the antiviral composition includes a moisturizer. The moisturizer can be a hydrophilic component including humectants such as propylene glycol, dipropylene glycol, polyethylene glycols, glycerol, sorbitol, alpha-hydroxy acids, urea, amino acids, ethoxylated amides, sodium pyrrolidone carboxylic acid and combinations thereof. Additionally, the moisturizer can be a hydrophobic occlusive component which helps to retain moisture including emollients such as mineral oil, squalene, petrolatum, cocoa butter, beeswax, jojoba oil, lanolin and derivatives, silicones, fatty acids, fatty alcohols, fatty acid esters, fatty alcohol esters, fatty acid triglycerides, and combinations thereof.

Certain materials including some humectants or emollients are particularly useful at providing safe and effective skin protection. Preferred skin protectants include allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

The present invention also provides methods of use of compositions of the present invention. In one embodiment, the present invention provides a method of preventing and/or treating an viral infection caused, or aggravated by, a microorganism on mammalian tissue, particularly the skin and/or mucous membrane in and surrounding the oral cavity. The method includes contacting the mucous membranes in and around the oral cavity with an antiviral composition of the present invention.

In other embodiments, the present invention provides methods for killing or inactivating microorganisms. Herein, to “kill or inactivate” means to render the microorganism ineffective by killing them (e.g., bacteria and fungi) or otherwise rendering them inactive (e.g., viruses). The present invention provides methods for inactivating enveloped viruses including but not limited to the viruses of the herpes family, such as Herpes Simplex I, Herpes Simplex II, Herpes Simplex VI, herpes zoster; poxviruses; corona viruses; orthomyxoviruses; paramyxoviruses; and togaviruses.

In certain embodiments, the composition of the present invention provides methods for killing bacteria and/or preventing bacterial infection for such as Staphylococcus spp., Streptococcus spp., Escherichia spp., Enterococcus spp., Pseudomonas spp. bacteria and combinations thereof, and more particularly Staphylococcus aureus (including antibiotic resistant strains such as methicillin resistant Staphylococcus aureus), Staphylococcus epidermidis, Escherichia coli (E. coli), Pseudomonas aeruginosa (Pseudomonas ae.), Streptococcus pyogenes, and combinations thereof which often are on or in the skin or mucosal tissue of a subject. The method includes contacting the microorganism with an antiviral composition of the present invention in an amount effective to kill one or more microorganisms (e.g., bacteria and fungi) or inactivate one or more microorganisms (e.g., viruses, particularly herpes virus).

In another embodiment, the present invention provides a method of topically treating a viral infection in mammals caused by the herpes family of viruses. Viral infections caused by the herpes family of viruses include cold sores, shingles, chicken pox, and genital herpes. The method includes contacting the affected area with an antiviral composition that includes: an effective amount of an antiviral lipid component that includes a (C7-C14) saturated fatty acid ester of propylene glycol, a (C8-C22) unsaturated fatty acid ester of a propylene glycol, or combinations thereof in an amount greater than 20 wt-%, and an organoleptic neutralizing agent.

In yet another embodiment, the present invention provides a composition useful for the topical treatment of an herpes viral infection and a method of topically treating said infection by contacting the affected area with an antiviral composition that includes an effective amount of an antiviral lipid component that includes a (C7-C14) saturated fatty acid ester of propylene glycol, a (C8-C22) unsaturated fatty acid ester of a propylene glycol, or combinations thereof, in combination with a organoleptic neutralizing agent.

The compositions of the present invention can also be used for providing residual antimicrobial efficacy on a surface that results from leaving a residue or imparting a condition to the surface (e.g., skin, mucosal tissue) that remains effective and provides significant antimicrobial activity. This in particular may reduce the infectiousness of exanthemas, skin rashes, and lesions caused by measles, cold sores, chickenpox, hand foot and mouth disease, rubella, and roseola, among others. Further, such compositions may be used to prevent secondary bacterial infections at a viral site.

### Definitions

The following terms are used herein according to the following definitions.

“Essential oil” means the volatile ethereal fraction obtained from a plant or plant part by a separation method.

“Organoleptic” means any sensory properties of a product, involving taste, colour, odour and feel. As used herein, the organoleptic characteristic of taste, and to a lesser extent smell, are the two organoleptic characteristics of most interest in the compositions described herein.

“External analgesic” means a topically applied compound that has an analgesic, anesthetic, or antipruritic effect by depressing cutaneous sensory receptors, or that has a topical counterirritant effect by stimulating cutaneous sensory receptors.

“Effective amount” means the amount of the antiviral lipid component and/or the enhancer component when in a composition, as a whole, provides an antimicrobial (including, for example, antiviral, antibacterial, or antifungal) activity that reduces, prevents, or eliminates one or more species of microbes such that an acceptable level of the microbe results. Typically, this is a level low enough not to cause clinical symptoms, and is desirably a non-detectable level.

It should be understood that (unless otherwise specified) the listed concentrations of all components are for “ready to use” or “as used” compositions.

“Moisturizer” refers to a material that will increase the level of hydration of skin, mucous membrane, wound, lesion, or scab.

A “humectant” is a polar hygroscopic material that increases hydration by drawing water from the environment to help retain water in the skin's upper layers.

An “emollient” is a hydrophobic material that provides softness, lubricity, and smoothness to the skin and often forms a thin occlusive film which increases hydration by reducing transepidermal water loss (TEWL).

“Stable” means physically stable or chemically stable, which are both defined in greater detail below.

“Enhancer” means a component that enhances the effectiveness of the antimicrobial lipid component such that when the composition less the antiviral lipid component and the composition less the enhancer component are used separately, they do not provide the same level of antimicrobial activity as the composition as a whole. For example, an enhancer component in the absence of the antiviral lipid component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the composition as a whole displays an activity that is greater than the sum of the activity of the composition less the enhancer component and the composition less the antiviral lipid component.

“Microorganism” or “microbe” refers to bacteria, yeast, mold, fungi, protozoa, mycoplasma, as well as viruses (including lipid enveloped RNA and DNA viruses).

“Antibiotic” means an organic chemical produced by microorganisms that has the ability in dilute concentrations to destroy or inhibit microorganisms and is used to treat infectious disease. This may also encompass semi-synthetic compounds that are chemical derivatives of the compound produced by microorganisms or synthetic compounds that act on very specific biochemical pathways necessary for the cell's survival.

“Antiseptic” means a chemical agent that kills pathogenic and non-pathogenic microorganisms. Antiseptics generally interfere more broadly with the cellular metabolism and/or the cell envelope.

“Mucous membranes,” “mucosal membranes,” and “mucosal tissue” are used interchangeably and refer to the surfaces of the nasal (including anterior nares, nasopharangyl cavity, etc.), oral (e.g., mouth including the inner lip, buccal cavity and gums), and other similar tissues. Examples include mucosal membranes such as buccal, gingival, and nasal mucosal membranes.

“Antiviral lipid” means an antiseptic having at least one alkyl or alkylene group having at least 6 carbon atoms, preferably at least 7 carbon atoms, even more preferably at least 8 carbon atoms, and has a hydrophile/lipophile balance (HLB) of at most 6.2, more preferably at most 5.8, and even more preferably at most 5.5. The antiviral lipid preferably has an HLB of at least 3, preferably at least 3.2, and even more preferably at least 3.4.

“Fatty” as used herein refers to a straight or branched chain alkyl or alkylene moiety having at least 6 carbon atoms, unless otherwise specified.

“Affliction” means a condition to a body resulting from sickness, disease, injury, bacterial colonization, etc.

“Treat” or “treatment” means to improve the condition of a subject relative to the affliction, typically in terms of clinical symptoms of the condition.

“Subject” and “patient” includes humans, sheep, horses, cattle, pigs, dogs, cats, rats, mice, or other mammals.

“Wound” refers to an injury to a subject which involves a break in the normal skin or mucosal tissue barrier exposing tissue below, which is caused by, for example, lacerations, surgery, burns, damage to underlying tissue such as pressure sores, poor circulation, and the like. Wounds are understood to include both acute and chronic wounds.

“Lesion” as used herein is an abnormal condition of a tissue (e.g., skin and/or mucous membrane) caused by a microbial (e.g., bacteria, viral, and/or fungal) infection.

The terms “comprises” and variations thereof do not have a limiting meaning where these terms appear in the description and claims.

As used herein, “a,” “an,” “the,” “at least one,” and “one or more” are used interchangeably. The term “and/or” means one or all of the listed elements (e.g., preventing and/or treating an affliction means preventing, treating, or both treating and preventing further afflictions).

Also herein, the recitations of numerical ranges by endpoints include all numbers subsumed within that range (e.g., 1 to 5 includes 1, 1.5, 2, 2.75, 3, 3.80, 4, 5, etc.).

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### DETAILED DESCRIPTION OF ILLUSTRATIVE EMBODIMENTS

The present invention provides antimicrobial (including, e.g., antiviral, antibacterial, and antifungal) compositions. These compositions include one or more antiviral lipids, such as, for example, a fatty acid ester of a polyhydric alcohol, a fatty ether of a polyhydric alcohol, a fatty alcohol ester of a hydroxyacid, or alkoxylated derivatives thereof (of either the ester or ether). These compositions also include one or more organoleptic neutralizing agents comprising compounds such as, for example, terpineols, alpha-pinene, borneol, borneol acetate, patchoulol, cineol, linalool, citronellal, and forskolin. The organoleptic neutralizing agents are preferably in the form of essential oils containing a major amount of the compounds such as those listed above.

Certain compositions also include one or more external analgesics, and/or one or more moisturizers. In certain embodiments, the moisturizer can be the same as the antiviral lipid component.

The compositions disclosed herein are useful for treating an infection caused by a herpes virus. The compositions, which include topical creams and ointments, are useful for treating topical skin infections caused by a herpes virus including but not limited to cold sores, shingles, and genital herpes. The formulations of this invention are useful for treating and preventing infections caused by a member of the herpes virus family.

The compositions are particularly useful for treating and preventing cold sores caused by the herpes simplex I virus. About 15-20% of the adult population in the United States suffers occasionally from painful open lesions on or near the lips caused by this virus. The compositions are also useful for treating shingles (caused by a herpes zoster virus) which is a painful rash of small blisters on a strip of skin anywhere on the body, most often on the trunk and buttocks. Animal models show that the formulations described herein perform equally as well as commercial antiviral prescription products, particularly 5% acyclovir ointment (commercially available from GlaxoSmithKline under the tradename ZOVIRAX). The formulations described herein further constitute antiseptics with a lower probability for developing antiviral resistance. Furthermore, the compositions can prevent the formation of a secondary bacterial infection in an open lesion or infection site. Hence, patients suffering with viral infections may be able to avoid other prophylactic antimicrobial treatments, such as oral antibiotics.

Such compositions adhere well to bodily tissues (i.e., mammalian tissues such as skin and mucosal tissue) and thus are very effective topically. Thus, the present invention provides a wide variety of uses of the compositions. Particularly preferred methods involve topical application, particularly to skin (e.g., skin lesions) and mucous membranes in and surrounding the oral cavity. Herein, such tissues are preferred examples of mammalian tissues.

Compositions described herein can be used to provide effective topical antimicrobial activity and thereby treat and/or prevent a wide variety of afflictions. For example, they can be used in the treatment and/or prevention of afflictions that are caused, or aggravated by, microorganisms (e.g., Gram positive bacteria, Gram negative bacteria, fungi, protozoa, mycoplasma, yeast, lipid-enveloped viruses) on skin and/or mucous membranes, such as those in the nose, mouth, or other similar tissues. Particularly relevant organisms that cause or aggravate such afflictions include viruses of the herpes family, such as Herpes Simplex I, Herpes Simplex II, Herpes Simplex VI, herpes zoster; poxvirus, corona virus, orthomyxovirus, paramyxovirus, and togavirus.

Compositions described herein can be used for the prevention and/or treatment of one or more microorganism-caused infections or other afflictions. In particular, the compositions can be used for preventing and/or treating cold sores.

The developmental stages of recurrent outbreaks caused by HSV-1 and/or HSV-2 are well known. The first, or prodromal stage, is characterized by normal appearance of skin accompanied by a tingling, burning, painful, or itching sensation. Subsequent stages include the formation of maculopapular lesions that develop into small, tense vesicles or blisters. The vesicles eventually break or collapse, with or without the formation of ulcers. Eventually, the lesion forms a crust. Overall, the lesion may last from seven to ten days.

Preferred compositions described herein can be used to treat outbreaks of lesions caused by HSV-1 and/or HSV-2. Application of the compositions can be applied at any stage of the outbreak of lesions to reduce the number of lesions and/or shorten the length of time of the outbreak. Application of the compositions during the prodromal stage may prevent or minimize the length or severity of the outbreak of lesions. In addition, the compositions may reduce the viral load at the infection site.

In one preferred embodiment, combining an organoleptic neutralizing agent comprising tea tree oil with an antiviral lipid component, such as propylene glycol monolaurate, provides a synergistic effect when treating type I Herpes Simplex viral (HSV I) infections. As shown in the Examples below, significant inhibition of lesion severity and size for this combination was observed on the 4^{th} and 5^{th} day after inoculation relative to tea tree oil alone or antiviral lipid component alone. The tea tree oil may also provide antibacterial benefit, reducing the chances of secondary infection of viral skin infections such as cold sores.

A combination of an antiviral lipid component and an organoleptic neutralizing agent can reduce the size of lesions caused by HSV I relative to similar formulations with just antiviral lipid component alone or an antimicrobial organoleptic neutralizing agent alone. Treatment of cold sores can therefore be accelerated, reducing both the pain and the unsightly sore that develops in the areas surrounding the oral cavity.

Preferred compositions contain an effective amount of antiviral lipid component to rapidly kill or inactivate microorganisms on skin, skin lesions, and mucosal membranes. Preferred compositions inactivate virions preventing transmission of an infectious virion from one person to another. Preferred compositions also have a generally low irritation level for skin, skin lesions, and mucosal membranes.

Preferred compositions described herein are substantive for relatively long periods of time to ensure adequate efficacy. For example, certain compositions remain at the site of application with antimicrobial activity for at least 4 hours and more preferably at least 8 hours.

In certain embodiments, the compositions may optionally include a penetration agent. A penetration agent is a compound that enhances the antiseptic diffusion into or through the skin or mucosal tissue by increasing the permeability of the tissue to the antimicrobial component and pharmacologically active agent, if present, to increase the rate at which the drug diffuses into or through the tissue. Examples of penetration agents are described in PCT Patent Application No. US2006/008953.

Preferred compositions described herein are physically stable. As defined herein “physically stable” compositions are those that do not significantly change due to substantial precipitation, crystallization, phase separation, and the like, from their original condition during storage at 23° C. for at least 3 months, and preferably for at least 6 months. Particularly preferred compositions are physically stable if a 10-milliliter (10-ml) sample of the composition when placed in a 15-ml conical-shaped graduated plastic centrifuge tube (Corning) and centrifuged at 3,000 revolutions per minute (rpm) for 10 minutes using a Labofuge B, model 2650 manufactured by Heraeus Sepatech GmbH, Osterode, West Germany (or similar centrifuge at 2275×g) has no visible phase separation in the bottom or top of the tube.

Preferred compositions described herein exhibit good chemical stability. This can be especially a concern with the antiviral fatty acid esters, which can often undergo transesterification, for example. Preferred compositions retain at least 85%, more preferably at least 90%, even more preferably at least 92%, and even more preferably at least 95%, of the antiviral lipid component after aging for 4 weeks at 40° C. (an average of three samples) beyond the initial 5-day equilibration period at 23° C. The most preferred compositions retain an average of at least 97% of the antiviral lipid component after aging for 4 weeks at 40° C. in a sealed container beyond the initial 5-day equilibration period at 23° C. The percent retention is understood to mean the weight percent of antiviral lipid component retained. This is determined by comparing the amount remaining in a sample aged (i.e., aged beyond the initial 5-day equilibration period) in a sealed container that does not cause degradation, to the actual measured level in an identically prepared sample (preferably from the same batch) and allowed to sit at 23° C. for five days. The level of antiviral lipid component is preferably determined using gas chromatography as described in the Aging Study Using Gas Chromatography test method method described in U.S. Patent Publication No. 2005/0089539-A1.

Generally, the compositions of this invention may be in one of the following forms:

A hydrophobic or hydrophilic ointment: The compositions are formulated with a hydrophobic base (e.g., petrolatum, thickened or gelled water insoluble oils, and the like) and optionally having a minor amount of a water soluble phase. Hydrophilic ointments generally contain one or more surfactants or wetting agents.

An oil-in-water emulsion: The compositions may be formulations in which the antiviral lipid component is emulsified into an emulsion comprising a discrete phase of a hydrophobic component and a continuous aqueous phase that includes water and optionally one or more polar hydrophilic material(s) as well as salts, surfactants, emulsifiers, and other components. These emulsions may include water-soluble or water-swellable polymers as well as one or more emulsifier(s) that help to stabilize the emulsion. These emulsions generally have higher conductivity values, as described in U.S. Pat. No. 7,030,203.

A water-in-oil emulsion: The compositions may be formulations in which the antiviral lipid component is incorporated into an emulsion that includes a continuous phase of a hydrophobic component and an aqueous phase that includes water and optionally one or more polar hydrophilic material(s) as well as salts or other components. These emulsions may include oil-soluble or oil-swellable polymers as well as one or more emulsifier(s) that help to stabilize the emulsion.

Thickened Aqueous gels: These systems include an aqueous phase which has been thickened by suitable natural, modified natural, or synthetic polymers as described below. Alternatively, the thickened aqueous gels can be thickened using suitable polyethoxylated alkyl chain surfactants that effectively thicken the composition as well as other nonionic, cationic, or anionic emulsifier systems. Preferably, cationic or anionic emulsifier systems are chosen since some polyethoxylated emulsifiers can inactivate the antiviral lipids especially at higher concentrations.

Hydrophilic gels: These are systems in which the continuous phase includes at least one water soluble or water dispersible hydrophilic component other than water. The formulations may optionally also contain water up to 20% by weight. Higher levels may be suitable in some compositions. Suitable hydrophilic components include one or more glycols such as polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like. One skilled in the art will recognize that the level of ethoxylation should be sufficient to render the hydrophilic component water soluble or water dispersible at 23° C. In most embodiments, the water content is less than 20%, preferably less than 10%, and more preferably less than 5% by weight of the composition.

### Antiviral Lipid Component

The antiviral lipid component is that component of the composition that provides at least part of the antiviral activity. That is, the antiviral lipid component has at least some antiviral activity for at least one virus. It is generally considered the main active component of the compositions described herein.

The antiviral lipids preferably have a hydrophile/lipophile balance (HLB) of at most 7.5, more preferably at most 5.8, and even more preferably at most 5.5. The antiviral lipids preferably have an HLB of at least 3, preferably at least 3.2, and even more preferably at least 3.4.

Preferred antiviral lipids are uncharged and have an alkyl or alkenyl hydrocarbon chain containing at least 7 carbon atoms.

In certain embodiments, the antiviral lipid component preferably includes one or more fatty acid esters of a polyhydric alcohol, fatty ethers of a polyhydric alcohol, fatty alcohol esters of a hydroxyacid, or alkoxylated derivatives thereof (of either or both of the esters and ether), or combinations thereof. More specifically and preferably, the antiviral lipid component is selected from the group consisting of a (C7-C14) saturated fatty acid ester of a polyhydric alcohol (preferably, a (C8-C12) saturated fatty acid ester of a polyhydric alcohol); a (C8-C22) unsaturated fatty acid ester of a polyhydric alcohol (preferably, a (C12-C22) unsaturated fatty acid ester of a polyhydric alcohol); a (C7-C14) saturated fatty ether of a polyhydric alcohol (preferably, a (C8-C12) saturated fatty ether of a polyhydric alcohol); a (C8-C22) unsaturated fatty ether of a polyhydric alcohol (preferably, a (C12-C22) unsaturated fatty ether of a polyhydric alcohol); a (C7-C14) saturated fatty alcohol monoester of a (C2-C8) hydroxycarboxylic acid (preferably, a (C7-C12) saturated fatty alcohol monoester of a (C2-C8) hydroxycarboxylic acid, more preferably, a (C8-C12) saturated fatty alcohol monoester of a (C2-C8) hydroxycarboxylic acid); a (C8-C22) mono- or poly-unsaturated fatty alcohol monoester of a (C2-C8) hydroxycarboxylic acid; an alkoxylated derivative of any of the foregoing; and combinations thereof. Various combinations of monoesters, diesters, monoethers, and diethers can be used in a composition of the present invention.

A fatty acid ester of a polyhydric alcohol is preferably of the formula R¹—C(O)—O—R², wherein R¹ is the residue of a (C7-C14) saturated fatty acid (preferably, a (C8-C12) saturated fatty acid), or a (C8-C22) unsaturated fatty acid (preferably, a C12-C22) unsaturated, including polyunsaturated, fatty acid) and R² is the residue of a polyhydric alcohol (typically and preferably, propylene glycol, although a wide variety of others can be used including glycerin, pentaerythritol, sorbitol, ethylene glycol, hexylene glycol, polyglycerols, etc.). The R² group includes at least one free hydroxyl group (preferably, residues of glycerin, propylene glycol, or sucrose). Preferred fatty acid esters of polyhydric alcohols are esters derived from C8, C9, C10, C11, and C12 saturated fatty acids.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. As is generally know, 18:1, for example, means the compound has 18 carbon atoms and 1 carbon-carbon double bond. Preferred unsaturated chains have at least one unsaturated group in the cis isomer form.

In certain preferred embodiments, the fatty acid monoesters that are suitable for use in the present composition include known monoesters of propylene glycol monolaurate, propylene glycol monocaprate, propylene glycol monocaprylate, and combinations thereof. Propylene glycol monoesters are preferred because of their hydrolytic stability, liquid form, and ability to permeate the skin.

A fatty ether of a polyhydric alcohol is preferably of the formula R³—O—R⁴, wherein R³ is a (C7-C14) saturated aliphatic group (preferably, a (C8-C12) saturated aliphatic group), or a (C8-C22) unsaturated aliphatic group (preferably, (C12-C22) unsaturated, including polyunsaturated, aliphatic group) and R⁴ is the residue of glycerin, sucrose, or propylene glycol. Preferred fatty ethers are monoethers of (C7-C14) alkyl groups (more preferably, (C8-C12) alkyl groups).

Exemplary fatty monoethers include, but are not limited to, laurylglyceryl ether, caprylglyceryl ether, caprylylglyceryl ether, laurylpropyleneglycol ether, caprylpropyleneglycol ether, and caprylylpropyleneglycol ether. Other fatty monoethers include glycerin and propylene glycol monoethers of oleyl (18:1), linoleyl (18:2), linolenoyl (18:3), and arachidonoyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

A fatty alcohol ester of a hydroxyl functional carboxylic acid preferably has the formula:

R¹—O—(—C(O)—R²—O)ₙH

wherein R¹ is the residue of a (C7-C14) saturated alkyl alcohol (preferably, a (C7-C12) saturated alkyl alcohol, more preferably, a (C8-C12) saturated alkyl alcohol) or a (C8-C22) unsaturated alcohol (including polyunsaturated alcohol), R² is the residue of a hydroxycarboxylic acid wherein the hydroxycarboxylic acid has the following formula:

R³(CR⁴OH)ₚ(CH₂)_{q}COOH

wherein: R³ and R⁴ are each independently H or a (C1-C8) saturated straight, branched, or cyclic alkyl group, a (C6-C12) aryl group, or a (C6-C12) aralkyl or alkaryl group wherein the alkyl groups are saturated straight, branched, or cyclic, wherein R³ and R⁴ may be optionally substituted with one or more carboxylic acid groups; p=1 or 2; and q=0-3; and n=1, 2, or 3. The R³ group may include one or more free hydroxyl groups but preferably is free of hydroxyl groups. Preferred fatty alcohol esters of hydroxycarboxylic acids are esters derived from branched or straight chain C8, C9, C10, C11, and C12 alkyl alcohols. The hydroxyacids typically have one hydroxyl group and one carboxylic acid group. The hydroxycarboxylic acid moiety can include aliphatic and/or aromatic groups. For example, fatty alcohol esters of salicylic acid are possible. As used herein, a “fatty alcohol” is an alkyl or alkylene monofunctional alcohol having an even or odd number of carbon atoms.

Exemplary fatty alcohol monoesters of hydroxycarboxylic acids include, but are not limited to, (C8-C12) fatty alcohol esters of lactic acid such as octyl lactate, 2-ethylhexyl lactate (PURASOLV EHL from Purac, Lincolnshire Ill., lauryl lactate (CHRYSTAPHYL 98 from Chemic Laboratories, Canton Mass.), lauryl lactyl lacate, 2-ethylhexyl lactyl lactate; and (C8-C12) fatty alcohol esters of 3-hydroxybutanoic acid, mandelic acid; gluconic acid, tartaric acid, and salicylic acid. Preferred fatty alcohol esters are C12 (or lauryl) alcohol esters.

The alkoxylated derivatives of the aforementioned fatty acid esters, fatty alcohol esters, and fatty ethers (e.g., one which is ethoxylated and/or propoxylated on the remaining alcohol group(s)) also have antimicrobial activity as long as the total alkoxylate is kept relatively low. In the case where the esters and ethers are ethoxylated, the total moles of ethylene oxide is preferably less than 5, and more preferably less than 2.

The fatty acid esters or fatty ethers of polyhydric alcohols or fatty alcohol esters of hydroxyacids can be alkoxylated, preferably ethoxylated and/or propoxylated, by conventional techniques. Alkoxylating compounds are preferably selected from the group consisting of ethylene oxide, propylene oxide, and mixtures thereof, and similar oxyrane compounds.

The compositions disclosed herein include one or more fatty acid esters, fatty alcohol esters, fatty ethers, alkoxylated fatty acid esters, alkoxylated fatty alcohol esters, or alkoxylated fatty ethers at a suitable level to produce the desired result. Such compositions preferably include a total amount of such material of greater than 5 percent by weight (wt-%), more preferably greater than 10 wt-%, even more preferably greater than 15 wt-%, even more preferably greater than 20 wt-%, and even more preferably at least 25 wt-%, based on the total weight of the “ready to use” or “as used” composition. In a preferred embodiment, they are present in a total amount of no greater than 95 wt-%, more preferably no greater than 90 wt-%, even more preferably no greater than 80 wt-%, and even more preferably no greater than 70 wt-%, based on the “ready to use” or “as used” composition. Certain compositions may be higher in concentration if they are intended to be diluted prior to use.

Preferred compositions that include one or more fatty acid monoesters, fatty monoethers, or alkoxylated derivatives thereof can also include a small amount of a di- or tri-fatty acid ester (i.e., a fatty acid di- or tri-ester), a di- or tri-fatty ether (i.e., a fatty di- or tri-ether), or alkoxylated derivative thereof. Preferably, such components are present in an amount of no more than 50 wt-%, more preferably no more than 40 wt-%, even more preferably no more than 25 wt-%, even more preferably no more than 15 wt-%, even more preferably no more than 10 wt-%, even more preferably no more than 7 wt-%, even more preferably no more than 6 wt-%, and even more preferably no more than 5 wt-%, based on the total weight of the antiviral lipid component. For example, for monoesters, monoethers, or alkoxylated derivatives of glycerin, preferably there is no more than 15 wt-%, more preferably no more than 10 wt-%, even more preferably no more than 7 wt-%, even more preferably no more than 6 wt-%, and even more preferably no more than 5 wt-% of a diester, diether, triester, triether, or alkoxylated derivatives thereof present, based on the total weight of the antiviral lipid components present in the composition. However, as will be explained in greater detail below, higher concentrations of di- and tri-esters may be tolerated in the raw material if the formulation initially includes free glycerin because of transesterification reactions.

Although in some situations it is desirable to avoid di- or tri-esters as a component of the starting materials, it is possible to use relatively pure tri-esters in the preparation of certain compositions of the present invention (for example, as a hydrophobic component) and have effective antimicrobial activity.

### Organoleptic Neutralizing Agent

Compositions of the claimed invention also include one or more organoleptic neutralizing agents. The organoleptic neutralizing agent is a compound or composition that improves the organoleptic properties of the antiviral composition.

The antiviral lipids disclosed herein, and particularly the fatty acid esters, are known to have a distinct bitter and/or soapy taste that lingers in the mouth for prolonged periods when applied on or near the oral cavity. The taste can provoke a gag, wretch, or shudder response in some individuals.

The organoleptic neutralizing agent may itself have unacceptable organoleptic properties, such as for example, tea tree oil and myrtle oil. However, when combined with the antiviral lipid component in the composition, the resulting composition has acceptable organoleptic properties, e.g., taste.

The organoleptic neutralizing agent is believed to shift or counteract the taste effect of the antiviral lipid component. The mechanism of how this occurs is not known, as the organoleptic neutralizing agent does not necessarily have acceptable taste itself. Thus, the organoleptic neutralizing agent is not necessarily acting as a masking agent. Masking agents are known to function by overwhelming the senses, e.g., TENS analogy, and includes organoleptic reactions of both taste and smell. Similarly, the organoleptic neutralizing agent is not necessarily acting as a flavorant, which typically functions by masking a negative taste with a stronger better taste that is a recognizable flavor but typically erodes with the underlying bad taste returning to the oral cavity. Rather, the organoleptic neutralizing agent appears to shift the organoleptic properties of the overall composition, irrespective of the organoleptic properties of the individual components. Further, the resulting composition doesn't taste like either of the individual components, i.e., the antiviral lipid and the organoleptic neutralizing agent.

The organoleptic neutralizing agents typically comprise compounds with a structure selected from the group consisting of a hydrocarbon monoterpene of formula C₁₀H₁₆ selected from an acyclic compound, a monocyclic, or a bicyclic compound; an oxygen containing monoterpene of formula C₁₀H₁₈O selected from an acyclic compound, a monocyclic compound, or a bicyclic compound; an oxygen containing acyclic monoterpene of formula C₁₀H₂₀O; the sesquiterpene patchoulol; the diterpene forskolin; the acetate esters of the above oxygenated compounds that are alcohols, and mixtures thereof. Compounds containing the structures listed above include myrcene, limonene, beta-phellandrene, alpha-terpinene, gamma-terpinene, alpha-pinene, beta-pinene, geraniol, linalool, citronellal, terpinen-4-ol, borneol, 1,8-cineol, isoborneol, and citronellol.

The organoleptic neutralizing agents are typically added to the composition in the form of essential oils. The essential oils that function as organoleptic neutralizing agents contain a major amount of one or more of the compounds listed above. Essential oils containing these compounds include but are not limited to tea tree oil, rosemary oil, lavender, pine oil, myrtle, eucalyptus, citronella, patchouli, and coleus extract oil.

In a preferred embodiment, the essential oils, many of them with antiseptic properties, when added to the compositions disclosed herein at 2% by weight modified the taste in a way that effectively overcame the soapy taste of the propylene glycol monolaurate in the antiviral composition. These compounds may be relatively water insoluble and thus it may be preferred to formulate these compounds in the presence of a hydrophobic component and/or an emulsifier/surfactant, in an emulsion (water in oil or oil in water), or in a hydrophilic vehicle.

These organoleptic neutralizing agents are typically added to the formulations at of at least 0.5% by weight, more preferably 1% by weight and most preferably 2% by weight. The organoleptic neutralizing agents are typically added to the formation at no more than 8%, more preferably no more than 6% by weight, and most preferably no more than 4% by weight. In some embodiments, the compositions are those based on hydrophobic vehicles (such as petrolatum) with an optional hydrophilic component and/or water in oil emulsions. The pH of compositions typically range from 3 to 9.

The compositions include one or more organoleptic neutralizing agents present in an amount sufficient to neutralize the taste of a antiviral lipid formulation. When added in the form of essential oils, such compositions preferably include a total amount of essential oils of at least 0.2 percent by weight (wt-%), more preferably at least 0.25 wt-%, even more preferably at least 0.35 wt-%, even more preferably at least 0.5 wt-%, and even more preferably at least 1, 2, or even 3 wt-%, based on the total weight of the “ready to use” or “as used” composition. In a preferred embodiment, the organoleptic neutralizing agents are present in a total amount of no greater than 20 wt-%, more preferably no greater than 15 wt-%, even more preferably no greater than 10 wt-%, and even more preferably no greater than 5 wt-%, based on the “ready to use” or “as used” composition.

In most embodiments, the ratio of antiviral lipid component to essential oil is less than 50 to 1, and more preferably less than 25 to 1, to affect the organoleptic properties of the antiviral composition and provide an acceptable taste. In a preferred embodiment, the ratio of antiviral lipid component to essential oil is 12 to 1.

When delivered in the form of essential oil, the weight of the compounds identified above, either alone or in combination in the essential oil, should preferably comprise at least 40 wt-% based on the total weight of essential oil, and more preferably at least 50 wt-%. When the compounds are used in their pure form, the corresponding amount necessary to modify the organoleptic properties of the antiviral composition would be lower based on the preferred amounts of organoleptic component as outlined above.

### External Analgesics

Safe and effective external analgesics include FDA-approved non-steroidal anti-inflammatories, local anaesthetics, topical steroids and the like. Preferred analgesics include amines and “caine’ types; alcohols and ketones; antihistamines; hydrocortisone preparations; and mixtures thereof. Preferred amine and “caine” type external analgesics include benzocaine, butamben picrate, dibucaine (or dibucaine HCl), dimethisoquin HCl, dyclonine HCl, lidocaine (or lidocaine HCl), pramoxine HCl, tetracaine (or tetracaine HCl), prilocaine and mixtures thereof, such as EMLA (an eutectic mixture of local anaesthetic comprised of 2.5% lidocaine and 2.5% prilocaine). Preferred alcohol and ketone type external analgesics include benzyl alcohol, camphor, camphorated metacresol, juniper tar, menthol, phenol, phenolate sodium, resorcinol, and mixtures thereof. Preferred antihistamine type external analgesics include diphenhydramine HCl, tripelennamine HCl, and mixtures thereof. Preferred hydrocortisone preparations include hydrocortisone, hydrocortisone acetate, and mixtures thereof. Mixtures of external analgesics from more than one type are also useful.

When used in an appropriate wt-%, they temporary relieve the symptoms, such as pain, inflammation or itch associated with a viral infection. Preferred amounts of amine and “caine” type external analgesics include 5 to 20 wt-% benzocaine, 1 wt-% butamben picrate, 0.25 to 1 wt-% dibucaine (or dibucaine HCl), 0.3 to 0.5 wt-% dimethisoquin HCl, 0.5 to 1.0 wt-% dyclonine HCl, 0.5 to 5 wt-% lidocaine (or lidocaine HCl), 0.5 to 1 wt-% pramoxine HCl, 1 to 2 wt-% tetracaine (or tetracaine HCl), and mixtures thereof. Preferred amounts of alcohol and ketone type external analgesics include 10 to 33 wt-% benzyl alcohol, 0.1 to 3 wt-% camphor, camphorated metacresol (with 3 to 10.8 wt-% camphor and 1 to 3.6 wt-% metacresol), 1 to 5 wt-% juniper tar, 0.1 to 1 wt-% menthol, 0.5 to 1.5 wt-% phenol, 0.5 to 1.5 wt-% phenolate sodium, 0.5 to 3 wt-% resorcinol, and mixtures thereof. Preferred amounts of antihistamine type external analgesics include 1 to 2 wt-% diphenhydramine HCl, 0.5 to 2% tripelennamine HCl, and mixtures thereof. Preferred amounts of hydrocortisone preparations include 0.25 to 0.5 wt-% hydrocortisone, 0.25 to 0.5 wt-% hydrocortisone acetate, and mixtures thereof. Mixtures of external analgesics from more than one type are also useful.

For external analgesics, the Proposed Final Rulemaking for Fever Blister and Cold Sore Treatment Drug Products in the External Analgesic Drug Products for Over-the-counter Human Use Monograph, published by the United States Food and Drug Administration in the Federal Register, Volume 55, Number 21, Jan. 31, 1990, pages 3370 to 3383 details: a) amine and “caine”-type local anesthetics including 1) 5 to 20% benzocaine, 7) 0.5 to 4% lidocaine, 9) 0.5 to 1% pramoxine hydrochloride, 10) 1 to 2% tetracaine, and b) alcohols and ketones including 1) 10 to 33% benzyl alcohol, 2) 0.1 to 3% camphor, 6) 0.1 to 1% menthol, 7) 0.5 to 1.5% phenol, 10) 0.5 to 3% resorcinol. Combinations of “a” with “b” are also permitted as are blends of menthol and/or camphor with benzyl alcohol, phenol, camphor, or other category b materials. A special combination of 3 to 10.8% camphor with 4.7% phenol combined in a light mineral oil is permitted under the regulations.

### Moisturizers

Compostions of the present invention may include a moisturizer to increase the level of hydration of skin, mucous membrane, wound, lesion, or scab. The moisturizer can be a hydrophilic material such as humectants or the moisturizer can be a hydrophobic material such as emollients.

Hydrophilic moisturizers. Exemplary hydrophilic moisturizers include, but are not limited to, water, polyhydric alcohols, lower alkyl ethers, N-methylpyrrolidone, lower alkyl esters, urea, amino acids, ethoxylated amides, sodium pyrrolidone carboxylic acid, and the lower monohydroxy alcohols and hydroxy acids discussed below as enhancers, as well as combinations thereof. Thus, a lower monohydroxy alcohol can function as both a hydrophilic compound and an enhancer. Preferably, the hydrophilic components include polyhydric alcohols, lower alkyl ethers, and short chain esters. More preferably, the hydrophilic components include polyhydric alcohols.

Suitable polyhydric alcohols (i.e., organic compounds having more than one hydroxyl group) have a molecular weight of less than 500, preferably less than 400, and more preferably less than 200. Examples of polyhydric alcohols include, but are not limited to, glycerol, propylene glycol, dipropylene glycol, tripropylene glycol, polypropylene glycol, polyethylene glycol, diethylene glycol, pentaerythritol, trimethylolpropane, trimethylolethane, trimethylolbutane, sorbitol, mannitol, xylitol, panthenol, ethylene glycol adducts of polyhydric alcohol, propylene oxide adducts of polyhydric alcohol, 1,3-butanediol, dipropylene glycol, diglycerine, polyglycerine, erythritol, sorbitan, sugars (e.g., sucrose, glucose, fructose, mannose, xylose, saccharose, trehalose), sugar alcohols, and the like. Certain preferred polyhydric alcohols include glycols (i.e., those containing two hydroxyl groups), glycerin and propylene glycol. Certain other preferred polyhydric alcohols include sucrose, xylitol, mannitol, and sorbitol.

Ethers include materials such as dimethylisosorbide, polyethylene glycol and methoxypolyethylene glycols, block and random copolymers of ethylene oxide and propylene oxide, and laureth-4. Alkyl esters include triacetin, methyl acetate, methyl lactate, ethyl lactate esters, esters of polyethoxylated glycols, and combinations thereof.

In certain preferred embodiments, the hydrophilic components useful as moisturizers in the compositions described herein include those selected from the group consisting of glycols, glycerin, propylene glycol, and mixtures thereof. Most preferably, the hydrophilic component is selected to match the polyhydric alcohol portion of any fatty acid monoester of a polyhydric alcohol antiviral present. For example, if the antiviral lipid component selected is glycerol monolaurate (monolaurin), then the most preferred hydrophilic component would be glycerin. In this manner, any transesterification reaction that may occur with the solvent or vehicle (i.e., the component used in the greatest amount and referred to as a “vehicle”) does not produce an undesirable by-product. If there are other components in the composition that may esterify with hydroxylfunctional hydrophilic components, conditions are selected to minimize this occurrence. For example, the components are not heated together for extended periods of time, and/or the pH is close to neutral if possible, etc.

One or more hydrophilic materials may be used in the compositions at a suitable level to produce the desired result. In certain preferred embodiments, the hydrophilic component is present in a total amount of at least 0.1%, preferably at least 1 wt-%, more preferably at least 4 wt-%, and even more preferably at least 8 wt-%, based on the weight of the ready to use composition. In certain embodiments, higher levels of hydrophilic component may be employed. In these cases the hydrophilic component is present in a total amount of at least 10 wt-%, more preferably at least 20 wt-%, and even more preferably at least 25 wt-%.

In a preferred embodiment, the hydrophilic component is present in a total amount of no greater than 70 wt-%, preferably no greater than 60 wt-%, more preferably no greater than 40 wt-%, even more preferably no greater than 30 wt-%, based on the ready to use composition. When the hydrophilic component is present in the greatest amount it is referred to as a “vehicle.”

Hydrophobic Moisturizers. Exemplary hydrophobic moisturizers include, but are not limited to, short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH; (C2-C18) alkyl or (C6-C12) aryl esters of glycerol, pentaerythritol, ethylene glycol, propylene glycol, as well as polyethoxylated derivatives of these; (C12-C22) alkyl esters or (C12-C22) ethers of polypropylene glycol; (C12-C22) alkyl esters or (C12-C22) ethers of polypropylene glycol/polyethylene glycol copolymer; and polyether polysiloxane copolymers. Additional examples of hydrophobic moisturizers include cyclic dimethicones, including volatile cyclic silicones such as D4 and D5, polydialkylsiloxanes, polyaryl/alkylsiloxanes, silicone copolyols, cocoa butter, beeswax, jojoba oil, lanolin and derivatives, long chain (i.e., C8-C36) alkyl and alkenyl esters of long (i.e., C8-C18) straight or branched chain alkyl or alkenyl alcohols or acids, long chain (i.e., C8-C36) alkyl and alkenyl amides of long straight or branched chain (i.e., C8-C36) alkyl or alkenyl amines or acids; hydrocarbons including straight and branched chain alkanes and alkenes such as isoparaffins (e.g., isooctane, isododecane, isooctadecane, etc.), squalene, and mineral oil, polysiloxane polyalkylene copolymers, dialkoxy dimethyl polysiloxanes; (C12-C22) alkyl and (C12-C22) alkenyl alcohols, and petroleum derived alkanes such as isoparaffins, petrolatum, petrolatum USP, as well as refined natural oils (especially NF or USP grades) such as olive oil NF, cotton seed oil, castor oil, peanut oil, corn oil, seasame oil, safflower oil, soybean oil, and the like, and blends thereof. In certain preferred embodiments, the hydrophobic moisturizers useful in the compositions of the present invention include those selected from the group consisting of petrolatum USP and short chain (i.e., C1-C6) alkyl or (C6-C12) aryl esters of long (i.e., C8-C36) straight or branched chain alkyl or alkenyl alcohols or acids and polyethoxylated derivatives of the alcohols; short chain (i.e., C1-C6) alkyl or (C6-C12) aryl esters of (C4-C12) diacids or (C4-C12) diols optionally substituted in available positions by —OH (such as diisopropyladipate, diisopropylsebacate); (C1-C9) alkyl or (C6-C12) aryl esters of glycerol, pentaerythritol, ethylene glycol, propylene glycol (such as glyceryl tricaprylate/caprate); and mixtures thereof.

### Skin Protectants

Certain materials including some humectants or emollients are particularly useful at providing safe and effective skin protection. When used in appropriate wt-%, they temporarily protect injured or exposed skin or mucous membrane surfaces from harmful or annoying stimuli, and may help provide relief to such surfaces. Preferred skin protectants include 0.5 to 2 wt-% allantoin, 0.15 to 5 wt-% aluminum hydroxide gel, 1 to 25 wt-% calamine, 50 to 100 wt-% cocoa butter, 5 to 13.56 wt-% cod liver oil, at least 0.007 wt-% colloidal oatmeal, 1 to 30 wt-% dimethicone, 20 to 45 wt-% glycerin, 50 to 100 wt-% hard fat, 4 to 20 wt-% kaolin, 12.5 to 50 wt-% lanolin, 50 to 100 wt-% mineral oil, 30 to 100 wt-% petrolatum, sodium bicarbonate, 10 to 98 wt-% topical starch, 0.1 to 2 wt-% zinc acetate, 0.2 to 2 wt-% zinc carbonate, 1 to 25 wt-% zinc oxide, 0.13 to 0.5 wt-% aluminum acetate, 46 to 63 wt-% aluminum sulfate, and witch hazel. Further information concerning safe and effective skin protectants useful in compositions described herein is provided in the Proposed Final Rulemaking for Fever Blister and Cold Sore Treatment Drug Products in the Skin Protectant Drug Products for Over-the-counter Human Use Monograph, published by the United States Food and Drug Administration in the Federal Register, Volume 51, Number 21, Jan. 31, 1990, pages 3362 to 3370.

### Enhancer Component

Compositions described herein may optionally include an enhancer (and preferably a synergist) to enhance the antimicrobial activity especially against Gram negative bacteria, such as E. coli and Pseudomonas sp. The enhancer component may include an alpha-hydroxy acid, a beta-hydroxy acid, other carboxylic acids, a (C1-C4) alkyl carboxylic acid, a (C6-C12) aryl carboxylic acid, a (C6-C12) aralkyl carboxylic acid, a (C6-C12) alkaryl carboxylic acid, a phenolic compound (such as certain antioxidants and parabens), a (C1-C10) monohydroxy alcohol, a chelating agent, or a glycol ether (i.e., ether glycol) as described in U.S. Patent Publication No. 2005/0089539-A1. Various combinations of enhancers can be used if desired.

One or more enhancers may be used in the compositions of the present invention at a suitable level to produce the desired result. In a preferred embodiment, they are present in a total amount greater than 0.01 wt-%, more preferably in an amount greater than 0.1 wt-%, even more preferably in an amount greater than 0.2 wt-%, even more preferably in an amount greater than 0.25 wt-%, and most preferably in an amount greater than 0.4 wt-% based on the total weight of the ready to use composition. In a preferred embodiment, they are present in a total amount of no greater than 20 wt-%, based on the total weight of the ready to use composition. Such concentrations typically apply to alpha-hydroxy acids, beta-hydroxy acids, other carboxylic acids, chelating agents, phenolics, ether glycols, (C5-C10) monohydroxy alcohols. Generally, higher concentrations are needed for (C1-C4) monohydroxy alcohols.

In a preferred embodiment, the short chain (i.e., C1-C4) alcohols are present in a total amount of at least 10 wt-%, even more preferably at least 15 wt-%, even more preferably at least 20 wt-%, and even more preferably at least 25 wt-%, based on the total weight of the ready to use composition.

In a preferred embodiment, the (C1-C4) alcohols are present in a total amount of no greater than 90 wt-%, more preferably no greater than 70 wt-%, even more preferably no greater than 60 wt-%, and even more preferably no greater than 50 wt-%, based on the total weight of the ready to use composition.

### Surfactants

Compositions of the present invention optionally can include one or more surfactants to emulsify the composition and to help wet the surface and/or to aid in contacting the microorganisms. As used herein the term “surfactant” means an amphiphile (a molecule possessing both polar and nonpolar regions which are covalently bound) capable of reducing the surface tension of water and/or the interfacial tension between water and an immiscible liquid. The term is meant to include soaps, detergents, emulsifiers, surface active agents, and the like. The surfactant can be cationic, anionic, nonionic, or amphoteric. In preferred embodiments, the surfactant includes poloxamer, ethoxylated stearates, sorbitan oleates, high molecular weight crosslinked copolymers of acrylic acid and a hydrophobic comonomer, and cetyl and stearyl alcohols as cosurfactants.

A wide variety of conventional surfactants can be used; however, certain ethoxylated surfactants can reduce or eliminate the antimicrobial efficacy of the antiviral lipid component. The exact mechanism of this is not known and not all ethoxylated surfactants display this negative effect. For example, poloxamer (polyethylene oxide/polypropylene oxide) surfactants have been shown to be compatible with the antiviral lipid component, but ethoxylated sorbitan fatty acid esters such as those sold under the trade name TWEEN by ICI have not been compatible. It should be noted that these are broad generalizations and the activity could be formulation dependent. One skilled in the art can easily determine compatibility of a surfactant by making the formulation and testing for antimicrobial activity as described in U.S. Patent Publication No. 2005/0089539-A1. Combinations of various surfactants can be used if desired.

It should be noted that certain antiviral lipid components are amphiphiles and may be surface active. For example, certain antiviral alkyl monoglycerides described herein are surface active. For embodiments containing both an antiviral lipid component and a surfactant, the antiviral lipid component is considered distinct from a “surfactant” component.

### Thickeners

For certain applications, it may be desirable to formulate the antiviral lipid in compositions that are thickened with soluble, swellable, or insoluble organic polymeric thickeners such as natural and synthetic polymers including polyacrylic acids, poly(N-vinyl pyrrolidones), cellulosic derivatives, and xanthan or guar gums or inorganic thickeners such as silica, fumed silica, precipitated silica, silica aerogel and carbon black, and the like; other particle fillers such as calcium carbonate, magnesium carbonate, kaolin, talc, titanium dioxide, aluminum silicate, diatomaceous earth, ferric oxide and zinc oxide, clays, and the like; ceramic microspheres or glass microbubbles; ceramic microspheres suc as those available under the tradenames “ZEOSPHERES” or “Z-LIGHT” from 3M Company, St. Paul, Minn. The above fillers can be used alone or in combination in the compositions described herein.

### Optional Additives

Compositions of the present invention may additionally employ adjunct components conventionally found in cosmetic and pharmaceutical compositions as known in the art. Thus, for example, the compositions may contain additional compatible pharmaceutically active materials for combination therapy (such as supplementary antimicrobials, anti-parasitic agents, antipruritics, astringents, healing promoting agents, steroids, non-steroidal anti-inflammatory agents, or other anti-inflammatory agents), or may contain materials useful in physically formulating various dosage forms of the present invention, such as excipients, dyes, pigments, perfumes, fragrances, lubricants, thickening agents, stabilizers, skin penetration enhancers, preservatives, film forming polymers, or antioxidants. The compositions may also contain vitamins such as vitamin B, vitamin C, vitamin E, vitamin A, and derivates thereof.

It will also be appreciated that additional antiseptics, disinfectants, antiviral agents, or antibiotics may be included and are contemplated. These include, for example, addition of metals such as silver, copper, zinc; iodine and iodophors; chlorhexidine and its various salts such as chlorhexidine digluconate; polyhexamethylenebiguanide, parachlorometaxylenol, triclosan, antimicrobial quaternary amines including benzethonium chloride, benzalkonium chloride, and polymeric quaternary amines, “azole” antifungal agents including clotrimazole, miconazole, econazole, ketoconazole, and salts thereof; and the like. Antibiotics such as neomycin sulfate, bacitracin, mupirocin, polymyxin, rifampin, tetracycline, and the like, also may be included. Preferred compositions, however, are free of antibiotics due to the chance of resistance formation. Antiviral agents include, but are not limited to acyclovir, valacylovir, Pencidovir, and famcidovir.

It will be appreciated by the skilled artisan that the levels or ranges selected for the required or optional components described herein will depend upon whether one is formulating a composition for direct use, or a concentrate for dilution prior to use, as well as the specific component selected, the ultimate end-use of the composition, and other factors known to the skilled artisan.

Many of the compositions of the present invention have exceptional broad spectrum antimicrobial activity and thus are generally not terminally sterilized but if necessary may be sterilized by a variety of industry standard techniques. For example, it may be preferred to sterilize the compositions in their final packaged form using electron beam. It may also be possible to sterilize the sample by gamma radiation or heat. Other forms of sterilization may be acceptable. It may also be suitable to include preservatives in the formulation to prevent growth of certain organisms. Suitable preservatives include industry standard compounds such as PARABENS (methyl, ethyl, propyl, isopropyl, isobutyl, etc); 2-bromo-2 nitro-1,3-diol; 5-bromo-5-nitro-1,3-dioxane; chlorobutanol; diazolidinyl urea; iodopropynyl butylcarbamate; phenoxyethanol; halogenated cresols; methylchloroisothiazolinone and the like, as well as combinations of these compounds.

### Formulations and Methods of Preparation

The compositions of the present invention preferably adhere well to mammalian tissues (particularly, skin, mucosal tissue, and wounds), in order to deliver the antiviral to the intended site over a prolonged period even in the presence of perspiration. The component in the greatest amount (i.e., the vehicle) in the formulations of the invention may be any conventional vehicle commonly used for topical treatment of human or animal skin. As described further below, the hydrophobic ointments and the oil-in-water emulsions, which can take the form of a cream or lotion, are preferred embodiments of the present invention.

The formulations are typically selected from one of the following types:

(1) A hydrophobic ointment: The compositions are formulated with a hydrophobic base (e.g., petrolatum, thickened or gelled water insoluble oils, and the like) and optionally have a minor amount of a water soluble phase.

The hydrophobic ointment is an anhydrous or nearly anhydrous formulation with a hydrophobic vehicle. Typically the components of the ointment are chosen to provide a semi-solid consistency at room temperature which softens or melts at skin temperature to aid in spreading. Suitable components to accomplish this include low to moderate amounts of natural and synthetic waxes, for example beeswax, carnuba wax, candelilla wax, ceresine, ozokerite, microcrystalline waxes, and paraffins. Viscous semi-crystalline materials such as petrolatum and lanolin are useful in higher amounts. The viscosity of the ointment can also be adjusted with oil phase thickeners including hydrophobically modified clays.

In certain preferred embodiments of the present invention, the compositions are chosen to spread easily and absorb relatively rapidly into the epidermis. This rapid absorption is especially desirable when the composition is used to treat cold sores around the mouth. Rapid absorption is achieved by minimizing the amount of high melting waxes used and limiting the use of non-polar hydrocarbon materials such as petrolatum and mineral oil. Many of the preferred external analgesics and skin protectant materials described earlier are soluble in hydrophobic vehicles, particularly in the presence of the somewhat polar antiviral lipid component.

For materials that are not readily soluble, such as allantoin, or some of the enhancers, they can be suspended as solids in the ointment, or can be solubilized with a small amount of a hydrophilic component. For example, when formulating with organic acid enhancers or certain solid surfactants in petrolatum many enhancers and surfactants will dissolve into the petrolatum at temperatures above 85° C.; however, upon cooling, the enhancer and/or surfactant crystals or precipitates back out of solution making it difficult to produce a uniform formulation. If at least 0.1%, and preferably at least 1.0%, more preferably at least 2%, and most preferably at least 3 wt-%, of a hydrophilic compound (e.g., a glycol) is added, a stable formulation can be obtained. It is believed that these formulations produce an emulsion in which the enhancer and/or surfactant is dissolved, emulsified, or dispersed in the hydrophilic component which is emulsified into the hydrophobic component(s). These compositions are stable upon cooling and centrifuging.

Furthermore, it is believed that incorporation of the hydrophilic component in the formulation improves the antimicrobial activity. The mechanism for this is unknown; however, it may speed the release of the enhancer component and/or the antiviral lipid component.

The water content of these formulations is preferably less than 20%, preferably less than 10 wt-%, more preferably less than 5 wt-%, and even more preferably less than 2 wt-%, in order to minimize hydrolysis of any ester based antiviral lipid present.

Furthermore, it has been found that it is particularly desirable to use either glycerin or propylene glycol as the hydrophilic component in the hydrophobic ointment when the antiviral lipid component selected includes an ester. It is most preferred to use a hydrophilic compound that is identical to the glycol portion of the antiviral lipid, e.g., propylene glycol with the propylene glycol esters and glycerin with the glycerin esters. In this manner, transesterification of the antiviral lipid ester with the hydrophilic compound will not result in additional chemical species present. In fact, there is some evidence to show that use of glycerol monolaurate (95% pure), when formulated with glycerin as a hydrophilic compound, results in formation of additional glycerol monolaurate due to transesterification of the diester with the glycerin to produce two moles of the monoester. For this reason, it may be possible to initially formulate with lower grade glycerin ester that contains considerable levels of diester present, as long as it transesterifies during manufacture and/or storage to produce a formulation that includes less than 15% diester and preferably less than 5% diester based on the total weight of antiviral lipid component present.

These formulations can be relatively easily manufactured by first heating the hydrophobic component to 85° C., adding in the skin protectant if different from the hydrophobic component, surfactant, or hydrophilic component, cooling to 65° C., and adding the external analgesic, organoleptic neutralizing agent, and antiviral lipid component above its melting point. Alternatively, the enhancer component when used can be predissolved in the hydrophilic component (optionally along with the surfactant) and added to the hydrophobic component either before or after addition of the antiviral lipid component. If either the antiviral lipid component or the hydrophobic component is a solid at room temperature this is done at the minimum temperature necessary to melt all components. Exposure of ester-containing antiviral lipid components to enhancers that include either acid or ether groups to elevated temperatures for extended periods of time should be avoided to prevent transesterification reactions (unless transesterification reactions when utilizing lower purity fatty acid esters in combination with glycol hydrophilic components to produce the monoesters as discussed above).

The viscosity of these formulations intended for use on skin is preferably at least 500 centipoise (cps), more preferably at least 1,000 cps, and even more preferably at least 10,000 cps. The viscosity can be measured by the Viscosity Test as described in U.S. Patent Publication No. 2005/0089539-A1.

Similarly the viscosity and/or melt temperature can be enhanced by either incorporating a crystalline or semicrystalline hydrophobic material such as a higher melting petrolatum, addition of an insoluble filler/thixotrope, or by addition of a polymeric thickener (e.g., a polyethylene wax in a petrolatum vehicle). Polymeric thickeners may be linear, branched, or slightly crosslinked. It is important for comfort that the formulations are relatively soft and that they spread easily to allow easy application, especially over a wound, rash, or infected area.

(2) An oil-in-water emulsion: The compositions may be formulations in which the antiviral lipid component is emulsified into an emulsion comprising a discrete phase of a hydrophobic component and a continuous aqueous phase that includes water and optionally one or more polar hydrophilic material(s) as well as salts, surfactants, emulsifiers, and other components. These emulsions may include water-soluble or water-swellable polymers as well as one or more emulsifier(s) that help to stabilize the emulsion. These emulsions generally have higher conductivity values, as described in U.S. Pat. No. 7,030,203.

Antiviral lipid components of this invention can be formulated into oil-in-water emulsions in combination with the organoleptic neutralizing agent, such as essential oils. Particularly preferred compositions comprise an aqueous phase of at least 35 wt-%, preferably at least 40 wt-%, more preferably at least 45 wt-%, and most preferably at least 50 wt-%, by weight of the composition. As used herein the aqeuous phase includes all components which are soluble in water at 23° C. Several methods to produce stable oil-in-water emulsions are known to those skilled in the art including the use of stearate soaps, non-ionic surfactants, acrylates/C10-30 alkyl acrylate crosspolymers, and phase inversion emulsification. Generally speaking, the hydrophobic component (oil) is mixed in Container A along with any emulsifier(s) (optionally including polymeric emulsifiers) and heated to a temperature sufficient to ensure a homogenous composition and subsequent stable emulsion. For certain combinations of hydrophobic components, a homogeneous composition may result at room temperature and heating is not required. The temperature is typically raised to at least 60° C., preferably to at least 80° C., and more preferably to 100° C. or more.

In a separate Container B, the hydrophilic components are mixed, including one or more of the following: water, a hydrophilic component, enhancer(s), surfactant(s), and acids/bases to adjust the pH of the final composition. The contents of container B are heated to a temperature sufficient to ensure a stable final emulsion composition without significantly degrading any of the components, typically to a temperature greater than 40° C., preferably greater than 50° C., and more preferably greater than 60° C. While hot, container B is added to container A using a high shear mixer. The composition may be continuously mixed until cool (e.g., to a temperature of less than 40° C.) or it can be allowed to sit as long as the contents remain uniformly mixed. If the antiviral lipid component and/or the organoleptic neutralizing agent are heat sensitive, it is added with mixing during the cooling down period. If either component is not heat sensitive, it may be added to container A. The viscosity of these compositions may be adjusted by altering the levels of emulsifier; changing the ratio of water to oil phase; selection of the oil phase (e.g., select from an oil (hydrophobic component), which is more or less viscous); incorporation of a polymeric or particulate thickener, etc.

(3) A water-in-oil emulsion: The compositions may be formulations in which the antiviral lipid component is incorporated into an emulsion that includes a continuous phase of a hydrophobic component and an aqueous phase that includes water and optionally one or more polar hydrophilic materials(s) as well as salts or other components. These emulsions may include oil-soluble or oil-swellable polymers as well as one or more emulsifier(s) that help to stabilize the emulsion.

(4) Thickened aqueous gels: These systems include an aqueous phase which has been thickened by suitable natural, modified natural, or synthetic polymers. Alternatively, the thickened aqueous gels can be thickened using suitable polyethoxylated alkyl chain surfactants that effectively thicken the composition as well as other nonionic, cationic, or anionic emulsifier systems. Preferably, cationic or anionic emulsifier systems are chosen since some polyethoxylated emulsifiers can inactivate the antiviral lipids especially at higher concentrations.

(5) Hydrophilic gels: These are systems in which the continuous phase includes at least one water soluble hydrophilic component other than water. The formulations may optionally also contain water up to 20% by weight. Higher levels may be suitable in some compositions. Suitable hydrophilic components include one or more polyols such as glycerin, propylene glycol, butylene glycols, etc., polyethylene glycols (PEG), random or block copolymers of ethylene oxide, propylene oxide, and/or butylene oxide, polyalkoxylated surfactants having one or more hydrophobic moieties per molecule, silicone copolyols, as well as combinations thereof, and the like.

(6) Neat Compositions. The compositions of the present invention also may be delivered to the treatment site in a neat form or in a volatile solvent that rapidly evaporates to leave behind a neat composition. Such compositions may be solid, semi-solid, or liquid. In the case where the compositions are solid, the antimicrobial and/or the enhancer and/or the surfactant may optionally be microencapsulated to either sustain the delivery or facilitate manufacturing a powder, which is easily delivered. Alternatively, the composition can be micronized into a fine powder without the addition of other components or it may optionally contain fillers and other ingredients that facilitate powder manufacture. Suitable powders include, but are not limited to, calcium carbonate, calcium phosphate, various sugars, starches, cellulose derivatives, gelatin, and polymers such as polyethylene glycols.

When hydrophobic antimicrobial lipids are used, a method for micronizing a hydrophobic agent may be used wherein the hydrophobic agent is dissolved in an effective amount of a first solvent that is free of polymer (such as the method described in U.S. Pat. No. 6,746,635). The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

### Viscosity

Certain preferred compositions of the present invention have a viscosity of 500 Centipoise (cps) for ease of application topically. More preferably, compositions of the present invention have a viscosity of at least 1,000 cps, even more preferably at least 10,000 cps.

### Delivery Methods and Devices

Topical treatment regimens according to the practice of this invention include applying a safe and effective amount of the compositions described herein directly to the infected or at-risk skin, wound, or mucous membrane in or around oral cavity. Typically, the compositions are delivered to the skin and/or mucosal tissue in a manner that allows them to penetrate into the skin and/or mucosal tissue, as opposed to through the tissue into the blood stream. This concentrates the compositions locally at the site in need of treatment. Preferably treatment is started at the prodromal stage of the viral infection, prior to the development of a rash, sore or exanthema. Delivery can be accomplished by spraying, dipping, wiping, dropping, pouring, toweling, or the like, onto the area to be treated.

In the methods of the present invention, the compositions may be provided as a formulation suitable for delivery to mammalian tissue (e.g., skin and/or mucosal surfaces). Suitable formulations can include, but are not limited to, creams, gels, foams, ointments, lotions, balms, waxes, salves, solutions, suspensions, dispersions, water in oil or oil in water emulsions, microemulsions, pastes, powders, oils, lozenges, boluses, and sprays, and the like.

Various other modes of administration can be used as well known to one of skill in the art depending on the desired location for contact of the antiviral compositions of the present invention.

For application to skin or mucosal tissue, for example, the compositions may be applied directly to the tissue from a collapsible container such as a flexible tube, blow/fill/seal container, pouch, capsule, etc. In this embodiment, the primary container itself is used to dispense the composition directly onto the tissue or it can be used to dispense the composition onto a separate applicator. Other application devices may also be suitable including applicators with foam tips, brushes, and the like. Importantly, the applicator must be able to deliver the requisite amount of composition to the tissue. Therefore, in most instances applicator devices such as webs and swabs are coated on the applicator web at greater than 50% by weight of the dry web and preferably in excess of 100% by weight of the dry web. (On a swab this would include the weight only of the web and not the applicator stick.)

The collapsible containers may be made in a number of single layer, laminate, or coextruded constructions. Materials of construction may include polyolefins such as low, medium, or high density polyethylene including low and linear low density polyethylene, polypropylene, as well as copolymers of ethylene and/or propylene with other polar or non-polar comonomers; polyamides such as nylons; polyesters such as polyethylene terephalate, polybutyleneterephalate, polyethylene naphthalate; polyurethanes; polyacrylates; and the like. In some constructions it may be desirable to include a barrier material to prevent evaporation of one or more components of the formulation. Suitable barrier materials include polyesters (e.g., polyethylene terephthalate, polyethylene naphthalate, polybutylene terephalate, and the like), fluorinated layers such as polytetrafluoroethylene (PTFE, e.g., TEFLON), polyamides (e.g., nylon), chlorotrifluoroethylene (ACLAR), polyvinylidene fluoride, as well as copolymers of perfluorinated monomers with partially fluorinated monomers such as copolymers of tetrafluoroethylene/hexafluoropropylene/vinylidene fluoride (THV Fluorothermoplastic from Dyneon Company), polyvinylchloride, polyvinylidene chloride (PVDC, e.g., SARAN HB), ethylene vinyl alcohol (EVOH), polyolefins (e.g., polyethylene, high density polyethylene, polypropylene, and combinations thereof). Oriented and biaxially oriented polymers may be particularly preferred.

Particularly preferred barrier constructions include metallic foil barriers such as aluminum foil laminates, HDPE, PET, PETG, PEN laminates of polyester and polyolefin (in particular PET/HDPE or HDPE/PET/HDPE), laminates of PET and EVOH, biaxially oriented nylon, PVDC, Nylon/EVOH/Nylon (OXYSHIELD OUB-R), chlorotrifluoroethylene and laminates thereof, ceramic layer including silicon oxide (SiOₓ where x=0.5-2 and preferably 1-2) coated thermoplastics, and ceramic coated PET (CERAMIS available from CCL Container/Tube Division, Oak Ridge, N.J.).

The compositions of the present invention can be delivered from various substrates for delivery to the tissue. For example, the compositions can be delivered from a wipe or pad which when contacted to tissue will deliver at least a portion of the composition to the tissue.

The dose and frequency of application will depend on many factors including the condition to be treated, the concentration of antiviral lipid and enhancer, the microbe to be killed, etc. Typically, the compositions will be delivered in dosages of at least 10 milligrams per square centimeter (mg/cm²) of tissue, preferably at least 20 mg/cm² of tissue, more preferably at least 30 mg/cm² of tissue, and most preferably at least 50 mg/cm² of tissue, for most applications. Application can be made once, or several (e.g., 2-6) times daily for one or more days. Typically, the composition is applied 3 to 5 times/day for 1 to 7 days.

### Test Protocols

### Herpes Animal Model

Female 23-28 g (seven to eight weeks old) hairless mice were purchased from Charles River Labs (Wilmington, Mass.). They were quarantined one week prior to use, caged in shoebox-style polycarbonate cages with stainless steel tops, and fed standard mouse chow and tap water ad libitum.

Groups of 8 mice each were infected intradermally by lightly scratching the skin on the right shoulder and right hip of the animal using a 20 gauge hypodermic needle using 5 scratches horizontally within a 10 mm diameter square and then placing a drop of 1:10 dilution of the virus on the scratches and rubbing a virus into the scratches with the tip of the pipette.

The virus was a Type 1 herpes virus, strain KOS, initially obtained as a clinical isolate from Dr. Milan Fiala of Harbor General Hospital (Los Angeles, Calif.). It was passaged in Vero cells and titrated in mice prior to use in the experiment.

Topical treatment with all formulations described below began 4 hours after application of the virus, and continuing four times daily (every 6 hours) for 5 days. Treatment was achieved using a Teflon-coated metal spatula, rubbing approximately the same quantity of formulation into each lesion. A standard number of “rubs” was applied to each lesion. The animals were observed daily for the occurrence of death for 21 days.

Each lesion was assigned a score ranging from 0 (normal skin) to 4 (maximal lesion intensity) defined as “Lesion score”, and two measurements, a vertical length and a horizontal length, were taken of each lesion daily from days 1 through 10. These measurements were multiplied together and the “square area” recorded, defined as “Lesion Size”. The lesion scoring was done by technicians who are unaware of which group of animals they are examining in order to eliminate bias. The occurrence of new, satellite, lesions (e.g., another lesion located anywhere other than the site of the initial lesion) were also noted during this 10-day period. The mean of the lesions score and the lesions size was calculated based on the average of the measurements taken on the eight mice.

Two additional mice were used as toxicity controls. The shoulder of each of these animals was scratched as above but not exposed to virus. The formulation was rubbed into both the scratched shoulder and onto intact skin on the hip. These animals were weighed prior to initial treatment and again 18 hours after final treatment. They were also observed daily throughout the treatment for occurrence of skin irritation or other signs of toxicity. Deaths, if they occurred, were recorded daily for 21 days.

Differences in survivor numbers and numbers of satellite lesions were evaluated using Chi Square Analysis with Yates' correction for sample size. Inhibition of mean lesion size and delayed mean day to death were analyzed by t test. Lesion score differences were evaluated using Wilcoxon ranked sum analysis.

### Taste Test Protocol

The end of a cotton swab was used to transfer a small quantity of cream onto the middle of the tongue. The mouth was closed and the tongue pushed to the roof of the mouth while swallowing. The taste at the back of the tongue was noted for soapiness or bitterness both initially and after other tastes had faded. An overwhelming or persistent (beyond about 10 seconds) soapiness or bitterness was deemed unacceptable.

### EXAMPLES

Objects and advantages of this invention are further illustrated by the following examples, but the particular materials and amounts thereof recited in these examples, as well as other conditions and details, should not be construed to unduly limit the claims.

### Glossary of Components

| | Trade | | | |
|---|---|---|---|---|
| Material | name | CAS No. | Supplier | Address |
| L-menthol | None | 89-87-1 | Aldrich | Milwaukee, |
| crystals | | | Chemical | WI |
| Propylene | Capmul | 27194- | Abitec Corp. | Janesville, |
| glycol | PG-12 | 74-7 | | WI |
| monolaurate | | | | |
| Tea Tree Oil | | 8505-48- | Southern Cross | Lenox Head, |
| | | 9 or | Botanicals | Australia |
| | | 68674- | | |
| | | 73-4 | | |
| Poloxamer 185 | Pluracare | 106392- | BASF | Mt. Olive, |
| | P65 | 12-5 | | NJ |
| Benzocaine | | 94-09-7 | Merck | Rahway, NJ |
| USP | | | | |
| Propyl paraben | | 94-13-3 | Rita Corp | Woodstock, |
| | | | | IL |
| Deionized | None | | 3M lab, | St. Paul, |
| water | | | Millipore Unit | MN |
| Methyl paraben | | 99-76-3 | Rita Corp | Woodstock, |
| | | | | IL |
| Allantoin | | 97-59-6 | Spectrum | Gardenia, |
| | | | Chemicals | CA |
| Phenoxyethanol | | 122-99-6 | Sigma | St Louis, |
| | | | Chemicals | MO |
| Tocopherol | Vitamin E | 7695-91-2 | BASF | Mt. Olive, |
| acetate USP | acetate | | | NJ |
| carbomer | Ultrez 21 | | Noveon | Cleveland, |
| | | | | OH |
| Acrylates/C10–30 | Pemulen | | Noveon | Cleveland, |
| alkyl | TR-2 | | | OH |
| acrylate | | | | |
| crosspolymer | | | | |
| Glycerin USP | | 56-81-5 | P&G | Cincinnati, |
| | | | Chemicals | OH |
| Ethyl Oleate | none | 111-62-6 | ISP, Corp. | Somerset, |
| | | | | NJ |
| Dioctyl ether | Cetiol OE | | Cognis | |
| Cetyl alcohol | RITA CA | | Rita Corp. | Woodstock, |
| | | | | IL |
| 1N NaOH | | | | |

### Example 1 and Comparative Examples 1 and 2

Preparation: An oil-in-water emulsion was prepared by combining the liquid oil ingredients (A) at room temperature in a stainless steel beaker and adding powdered ingredients (B) to it with agitation to keep them slurried and suspended. A solution of water and glycerin (C) was added and moderate agitation continued for 20 minutes at room temperature. Neutralization by addition of NaOH (D) gives a thickened cream to which was added a solution of methyl and propyl parabens in phenoxyethanol (E). After stirring 20 minutes the resulting cream was transferred to a glass jar and sealed.

| Phase | | Ex 1 | Comp Ex 1 | Comp Ex 2 |
|---|---|---|---|---|
| A | Propylene glycol | 25 | 25 | 0 |
| | monolaurate | | | |
| A | Dioctyl ether | 0 | 0 | 25 |
| A | Menthol | 0.8 | 0.8 | 0.8 |
| B | Benzocaine USP | 5.0 | 0 | 5.0 |
| A | Ethyl oleate | 2.5 | 2.5 | 2.5 |
| A | Tocopherol acetate | 1.5 | 1.5 | 1.5 |
| A | Tea Tree Oil | 2.0 | 0 | 2.0 |
| A | Poloxamer 185 | 1.25 | 1.0 | 1.25 |
| C | Water | 53.11 | 58.6 | 53.11 |
| B | Ultrez 21 from Noveon | 0.1 | 0.1 | 0.1 |
| B | Pemulen TR2 from | 0.24 | 0.24 | 0.24 |
| | Noveon | | | |
| C | Glycerin | 5.0 | 5.0 | 5.0 |
| E | Phenoxyethanol | 0.6 | 0 | 0.6 |
| E | Propyl paraben | 0.1 | 0.16 | 0.1 |
| E | Methyl paraben | 0.2 | 0.10 | 0.2 |
| B | Allantoin | 1.0 | 1.0 | 1.0 |
| D | 1 N aqueous NaOH | 1.6 (to | 4 (to pH | ? (to pH |
| | | pH 7.6) | 7.6) | 7.6) |

### Comparative Example 3

A sample of a 5% acyclovir ointment was obtained (commercially available from GlaxoSmithKline under the tradename Zovirax).

### Examples 5-8

The formulations prepared in Example 1, Comparative Example 1, Comparative Example 2, and the purchased formulation of Comparative 3 were applied according to the Herpes Animal Model test protocol. The results of this experiment are summarized in Table 1.

TABLE 1
Effect of Tropical Treatment on HSV-1 Induced Lesions on Hairless Mice

| | | Tox Controls | | Infected, Treated Mice | | | | |
|---|---|---|---|---|---|---|---|---|
| | | | Mean | | | Day 7 | Day 7 | |
| | | | Weight | | Mean | Mean | Mean | Total |
| | | | Change^{a} | | Day to | Lesion | Lesion | Satelite |
| EX | Compound | Surv/Total | (g) | Surv/Total | Death^{b} ± SD | Scores^{c} ± SD | Size^{d} ± SD | Lesions^{e} |
| 5 | Example 1 | 2/2 | −0.3 | 7/9 | 10.5 | 1.0 ± 0.7 | 29.2 ± 34.1 | 0 |
| 6 | Comp Ex 1 | 2/2 | −1.8 | 5/9 | 12.0 | 0.8 ± 0.3 | 16.9 ± 12.2 | 1 |
| 7 | Comp Ex 2 | 2/2 | 2.2 | 6/8 | 7.8 | 1.0 ± 0.8 | 23.6 ± 26.3 | 2 |
| 8 | Comp Ex 3 | 2/2 | −0.8 | 8/8 | >21.0 | 0.0 ± 0.0 | 0.0 ± 0.0 | 0 |

^{a}Difference between initial weight and weight 18 hr after final treatment.
^{b}Mean day to death of mice dying prior to day 21.
^{c}Scores assigned from 0 (Normal) to 4 (maximal lesion intensity).
^{d}Vertical × horizontal measurement of two lesions (hip, shoulder) per mouse.
^{e}Separate lesions appearing later in the infection.

As seen in Table 1, the cutaneous herpes virus infection progressed through the neurons to induce a central nervous system infection that would kill 25% of the animals. The lesions were generally moderate in severity, with few achieving what was considered a 4+ (maximal) score; among the placebo-treated animals, usually the most severe lesion was approximately 2+. A score of 4+ was assigned to any animal that died of obvious infection. The size of the lesions in the untreated control mice varied rather extensively; on day 7, when the severity of the lesions was considered approximately at its maximum, the area sizes ranged from 4 mm² to 80 mm². Two satellite lesions were seen in these controls, occurring on days 9 and 10.

Tea tree oil, the essential oil of Melaleuca alternifolia, has been shown in in vitro studies to have virucidal activity against type 1 and type 2 herpesvirus. The unexpectedly good performance of the placebo (Comparative Example 2) in the present study could be explained by the presence of the tea tree oil.

### Comparative Examples 4-8 and Examples 9-17

Compositions were prepared with various essential oils according to the following process:

Process: Charge first six ingredients of phase A into a vessel and heat with mixing to 40-45 C to obtain a homogeneous solution. Stop heating and as solution cools add 2 wt-% natural essential oil (based on the final weight of the composition) and suspend the remaining phase A ingredients (allantoin, TR2 and Ultrez) in the oil phase by agitating at sufficient speed. Charge Phase B ingredients (glycerin and water) and mix for 60 to 70 minutes to hydrate the carbomers. Phase C: Charge 1 molar NaOH portionwise to pH 7.0 (2.55 parts required). Continue to mix the now thickened cream, adding Phenonip XB and diluting to 100 parts with water, mixing well. The resulting formulations had Brookfield viscosities in the range of 20,000 to 45,000 cps (spindle 4, 12 rpm).

| Ingredient | Amount |
|---|---|
| PHASE A | |
| Ethyl oleate | 2.5 |
| Propylene glycol monolaurate | 25.0 |
| Tocopheryl Acetate | 0.7 |
| Poloxamer 185 | 1.3 |
| Cetyl alcohol | 2.0 |
| Menthol USP | 1.0 |
| Nautral Essential Oil | 2.0 |
| Allantoin USP | 1.0 |
| Pemulen TR-2 | 0.25 |
| Carbomer | 0.10 |
| PHASE B | |
| Purified water | 54 |
| Glycerin USP | 5.0 |
| PHASE C | |
| Sodium hydroxide | QS to pH 5.5 to 7 |
| Methyl paraben (and) Ethyl Paraben | 1.0 |
| (and) Propyl Paraben (and) | |
| Phenoxyethanol (Phenonip XB from | |
| ISP) | |
| Purified water | QS to 100 |

### Results of the taste test protocol on the formulations charged with various natural essential oils is shown Table 2.

| Ex. No. | Essential Oil | Taste | Major Chemical components |
|---|---|---|---|
| Comp. 4 | Clove oil | Unacceptable | 70 to 85% Eugenol, 5 to 15% Eugenyl |
| | | | acetate, 5 to 12% β-caryophyllene |
| Comp. 5 | Atlas Cedarwood | Unacceptable | 7 to 16% α-himachalene, 5 to 9% γ- |
| | oil | | himachalene, 23 to 40% β-himachalene 5 |
| | | | to 29% (E)-α-atlantone |
| Comp. 6 | Roman Chamomile | Unacceptable | 21% 3-methylpentyl isovalerate, 16% |
| | | | methallyl angelate, 12% 3-methylpentyl |
| | | | isobutyrate, 8% 3-methylbutyl angelate |
| Comp. 7 | Vanillin | Unacceptable | 100% 4-hydroxy3-methoxybenzaldehyde |
| Comp. 8 | Thyme oil | Unacceptable | 40% thymol, 30% para-cymene |
| Ex. 9 | Tea Tree Oil | Acceptable | 40% α-terpinene-4-ol, 10% α-terpinene, |
| | | | 20% γ-terpinene |
| Ex 10 | Rosemary | Acceptable | 30% 1,8-cineol, 16 to 20% borneol, 7% |
| | | | bornyl acetate, 25% α-pinene |
| Ex 11 | Lavender | Acceptable | 30 to 55% linalyl acetate, 20 to 35% |
| | | | linalool |
| Ex 12 | Pine oil | Acceptable | 19% α-pinene, 17% β-pinene, 14% |
| | | | myrcene, 29% β-phellandrene |
| Ex 13 | Myrtle | Acceptable | 14% α-pinene, 11% cineol, 20% linalool, |
| | | | 23% limonene |
| Ex 14 | Eucalyptus | Acceptable | 74% 1,8-cineole, 11% α-terpineol, 45% |
| | | | limonene |
| Ex 15 | Citronella | Acceptable | 32% citronella, 20% geraniol, 14.5% |
| | | | citronellol |
| Ex 16 | Patchouli | Acceptable | patchoulol |
| Ex 17 | Coleus Extract oil | Acceptable | forskolin |

Generally, the essential oils containing mono-aromatic terpenoids such as eugenol, thymol, carvacrol and vanillin failed to neutralize the unacceptable (bitter or soapy) taste of the formula. In addition, those essential oils comprised of esters of angelic acid (such as Roman Chamomile) failed to neutralize the taste sufficiently.

On the other hand, those containing monocyclic partially saturated rings, such as terpineol, bridged carbocycles such as alpha-pinene, borneol, borneol acetate, and patchoulol, and the bridged oxide cineol gave acceptable palatability. In addition, acyclic monoterpenes such as linalool and citronellal and the tricyclic diterpenoid forskolin effectively neutralized the unacceptable taste.

In particular, the tea tree oil provided a terpene-like medicinal taste and smell that effectively neutralized the unacceptable taste of the propylene glycol monolaurate.

### Comparative Examples 9-12

Several commercial flavorings were added to the formulation but the compositions still had unacceptable taste as determined by the taste test protocol. These included Cola flavor, Honey flavor, Tea flavor, and Butter rum flavor from Belle Aire Fragrances, Mundelein, Ill.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. A method of treating a viral infection caused by the herpes virus in or on the skin or mucous membrane, the method comprising contacting the affected area with an antiviral composition comprising:
an antiviral lipid component present in an amount greater that 15 wt-% comprising a (C7-C 12) saturated fatty acid ester of a polyhydric alcohol, a (C8-C22) unsaturated fatty acid ester of a polyhydric alcohol, an alkoxylated derivative thereof, or combinations thereof, wherein the alkoxylated derivative has less than 5 moles of alkoxide per mole of polyhydric alcohol; and
an organoleptic neutralizing agent.

2. The method of claim 1, wherein the organoleptic neutralizing agent comprises a compound with a structure selected from the group consisting of a hydrocarbon monoterpene of formula C₁₀H₁₆ selected from an acyclic compound, a monocyclic compound, or a bicyclic compound; an oxygen-containing monoterpene of formula C₁₀H₁₈₀ selected from an acyclic compound, a monocyclic compound, or a bicyclic compound; an oxygen-containing acyclic monoterpene of formula C₁₀H_{200;} the sesquiterpene patchoulol; the diterpene forskolin; the acetate esters of the foregoing oxygenated compounds that are alcohols; and mixtures thereof.

3. The method of claim 1, wherein the organoleptic neutralizing agent comprises a compound selected from the group consisting of myrcene, limonene, beta phellandrene, alpha-terpinene, gamma-terpinene, alpha pinene, beta-pinene, geraniol, linalool, citronellal, terpinen-4-ol, bomeol, 1,8-cineol, isobomeol, and citronellol.

4. The method of claim 1, wherein the organoleptic neutralizing agent comprises an essential oil selected from the group consisting of tea tree oil, rosemary oil, lavender, pine oil, myrtle, eucalyptus, citronella, patchouli, and coleus extract oil.

5. The method of claim 4, wherein the essential oil comprises tea tree oil.

6. The method of claim 1, wherein the organoleptic neutralizing agent comprises an essential oil comprising a major amount of a compound selected from the group consisting of myrcene, limonene, beta-phellandrene, alpha-terpinene, gamma-terpinene, alpha-pinene, beta-pinene, geraniol, linalool, citronellal, terpinen-4-ol, bomeol, 1,8-cineol, isobomeol, and citronellol.

7. The method of claim 1, wherein the organoleptic neutralizing agent comprises a compound with a structure selected from the group consisting of a hydrocarbon monoterpene of formula C₁₀H₁₆ selected from an acyclic compound, a monocyclic compound, or a bicyclic compound; an oxygen containing monoterpene of formula C₁₀H₁₈O selected from an acyclic compound, a monocyclic compound, or a bicyclic compound; an oxygen containing acyclic monoterpene of formula C₁H₂₀O; the sesquiterpene patchoulol; the diterpene forskolin; the acetate esters of the foregoing oxygenated compounds that are alcohols, and mixtures thereof.

8. The method of claim 1, wherein the organoleptic neutralizing agent is present in an amount less than 5 wt% based on the total weight of the antiviral composition.

9. The method of claim 1 wherein the antiviral lipid component is present in an amount greater that 20 wt%.

10. (canceled)

11. The method of claim 1, further comprising a moisturizer.

12. The method of claim 11, wherein the moisturizer comprises a humectant, an emollient, and combinations thereof.

13. The method of claim 1 wherein the antiviral lipid component further comprises no greater than 15 wt%, based on the total weight of the antiviral lipid component, of a di- or tri-ester, alkoxylated derivative thereof, or combinations thereof.

14. The method of claim 1, further comprising an external analgesic.

15. The method of claim 14, wherein the external analgesic is selected from the group consisting of benzocaine, butamben picrate, dibucaine, dibucaine HCl, dimethisoquin HCl, dyclonine HCl, lidocaine, lidocaine HCl, pramoxine HCl, tetracaine, tetracaine HCl, benzyl alcohol, camphor, camphorated metacresol, juniper tar, menthol, phenol, phenolate sodium, resorcinol, diphenhydramine HCl, tripelennamine HCl, hydrocortisone, hydrocortisone acetate, and mixtures thereof.

16. The method of claim 1, further comprising a skin protectant.

17. The method of claim 16, wherein the skin protectant is selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

18. The method of claim 1 wherein the antiviral lipid component comprises an effective amount of an antiviral lipid component comprising a (C7-C 14) saturated fatty acid ester of propylene glycol, a (C8-C22) unsaturated fatty acid ester of propylene glycol, and combinations thereof.

19. The method of claim 1 wherein the antiviral lipid component comprises propylene glycol monolaurate, propylene glycol monocaprate, propylene glycol monocaprylate, or combinations thereof.

20. The method of claim 1 further comprising a surfactant.

21. A topical antiviral composition comprising:
an antiviral lipid component comprising a (C7-C 14) saturated fatty acid monoester of a polyhydric alcohol, a (C8-C22) unsaturated fatty acid monoester of a polyhydric alcohol, an alkoxylated derivative thereof, or combinations thereof, present in an amount greater than 5 wt- % based on the total weight of the composition; and
an organoleptic neutralizing agent.

22. The composition of claim 21, wherein the organoleptic neutralizing agent comprises a compound with a structure selected from the group consisting of a hydrocarbon monoterpene of formula C₁₀H₁₆ selected from an acyclic compound, a monocyclic compound, or a bicyclic compound; an oxygen containing monoterpene of formula C₁₀H₁₈O selected from an acyclic compound, a monocyclic compound, or a bicyclic compound; an oxygen containing acyclic monoterpene of formula C₁₀H₂₀O; the sesquiterpene patchoulol; the diterpene forskolin; the acetate esters of the above oxygenated compounds that are alcohols, and mixtures thereof.

23. The composition of claim 21, wherein the organoleptic neutralizing agent comprises a compound selected from the group consisting of myrcene, limonene, beta phellandrene, alpha-terpinene, gamma-terpinene, alpha pinene, beta-pinene, geraniol, linalool, citronellal, terpinen-4-ol, bomeol, 1,8-cineol, isoborneol, and citronellol.

24. The composition of claim 21, wherein the organoleptic neutralizing agent comprises an essential oil selected from the group consisting of tea tree oil, rosemary oil, lavender, pine oil, myrtle, eucalyptus, citronella, patchouli, and coleus extract oil.

25. The composition of claim 21, wherein the essential oil comprises tea tree oil.

26. The composition of claim 21, wherein the organoleptic neutralizing agent comprises an essential oil comprising a major amount of a compound selected from the group consisting of myrcene, limonene, beta phellandrene, alpha-terpinene, gamma-terpinene, alpha pinene, beta-pinene, geraniol, linalool, citronellal, terpinen-4-ol, borneol, 1,8-cineol, isoborneol, and citronellol.

27. The composition of claim 21, wherein the organoleptic neutralizing agent comprises an essential oil comprising a major amount of a compound with a structure selected from the group consisting of a hydrocarbon monoterpene of formula C₁₀H₁₆ selected from an acyclic compound, a monocyclic compound, or a bicyclic compound; an oxygen containing monoterpene of formula C₁₀H₁₈₀ selected from an acyclic compound, a monocyclic compound, or a bicyclic compound; an oxygen containing acyclic monoterpene of formula C₁₀H_{200;} the sesquiterpene patchoulol; the diterpene forskolin; the acetate esters of the above oxygenated compounds that are alcohols, and mixtures thereof.

28. The composition of claim 21, wherein the organoleptic neutralizing agent is present in an amount less than 5 wt-% based on the total weight of the antiviral composition.

29. The composition of claim 21, wherein the antiviral lipid component is present in an amount greater that 5 wt-%.

30. The composition of claim 21, wherein the antiviral lipid component is present in an amount greater that 15 wt-%.

31. The composition of claim 21, further comprising a moisturizer.

32. The composition of claim 31, wherein the moisturizer comprises a humectant, an emollient, and combinations thereof.

33. The composition of claim 21, wherein the antiviral lipid component further comprises no greater than 15 wt-%, based on the total weight of the antiviral lipid component, of a di- or tri-ether, alkoxylated derivative thereof, or combinations thereof.

34. The composition of claim 21, further comprising an external analgesic.

35. The composition of claim 24, wherein the external analgesic is selected from the group consisting of benzocaine, butamben picrate, dibucaine, dibucaine HCl, dimethisoquin HCl, dyclonine HCl, lidocaine, lidocaine HCl, pramoxine HCl, tetracaine, tetracaine HCl, benzyl alcohol, camphor, camphorated metacresol, juniper tar, menthol, phenol, phenolate sodium, resorcinol, diphenhydramine HCl, tripelennamine HCl, hydrocortisone, hydrocortisone acetate, and mixtures thereof.

36. The composition of claim 21, further comprising a skin protectant.

37. The composition of claim 26, wherein the skin protectant is selected from the group consisting of allantoin, aluminum hydroxide gel, calamine, cocoa butter, cod liver oil, colloidal oatmeal, dimethicone, glycerin, hard fat, kaolin, lanolin, mineral oil, petrolatum, sodium bicarbonate, topical starch, zinc acetate, zinc carbonate, zinc oxide, aluminum acetate, aluminum sulfate, and witch hazel.

38. The composition of claim 21 wherein the antiviral lipid component comprises an effective amount of an antiviral lipid component comprising a (C7-C 14) saturated fatty acid ester of propylene glycol, a (C8-C22) unsaturated fatty acid ester of propylene glycol, and combinations thereof

39. The composition of claim 21 wherein the antiviral lipid component comprises propylene glycol monolaurate, propylene glycol monocaprate, propylene glycol monocaprylate, or combinations thereof.

40. The composition of claim 21 further comprising a surfactant.

41. A method of treating herpes lesions on or in the skin or mucous membranes, the method comprising contacting the affected area with an antiviral composition comprising:
an effective amount of an antiviral lipid component comprising a (C7-C 12) saturated fatty acid ester of a polyhydric alcohol, a (C8-C22) unsaturated fatty acid ester of a polyhydric alcohol, an alkoxylated derivative thereof, or combinations thereof, wherein the alkoxylated derivative has less than 5 moles of alkoxide per mole of polyhydric alcohol; and
an organoleptic neutralizing agent.

42. The method of claim 41 wherein in the herpes lesion is present on mucosal tissue.

43. A method of killing or inactivating microorganisms, the method comprising contacting the microorganisms with the antiviral composition of claim 21.

44. The method of claim 43 wherein the microorganisms comprise one or more viruses and the antiviral composition is used in an amount effective to inactivate one or more viruses.

45. (canceled)
